(19) United States
(12) Patent Application Publication
(10) Pub. No.: US 2025/0002551 A1
(43) Pub. Date: Jan. 2, 2025

(54) INTERLEUKIN 7 AND INTERLEUKIN 21 FUSION PROTEINS

(71) Applicant: Allegheny Singer Research Institute, Pittsburgh, PA (US)

(72) Inventors: David L. Bartlett, Pittsburgh, PA (US); Zuqiang Liu, Wexford, PA (US)

(21) Appl. No.: 18/700,852

(22) PCT Filed: Oct. 12, 2022
(86) PCT No.: PCT/US2022/077946

§ 371 Date: Apr. 12, 2024

Related U.S. Application Data
(60) Provisional application No. 63/255,635, filed on Oct. 14, 2021.

(51) Int. Cl. C07K 14/54; A61K 38/00; A61P 35/00; C12N 15/86

(52) U.S. Cl. CPC C07K 14/5418; A61P 35/00; C07K 14/54; C12N 15/86; A61K 38/00; C07K 2319/00; C12N 2710/24132; C12N 2710/24143

(57) ABSTRACT

Provided herein are fusion proteins comprising (i) interleukin 7 (IL-7) or an IL-7 variant and (ii) IL-21 or and IL-21 variant. Also provided herein are nucleic acids encoding a fusion protein comprising (i) IL-7 or an IL-7 variant and (ii) IL-21 or and IL-21 variant, oncolytic viruses encoding a fusion protein comprising (i) IL-7 or an IL-7 variant and (ii) IL-2I or and IL-21 variant, and immune cells expressing a fusion protein comprising (i) IL-7 or an IL-7 variant and (ii) IL-21 or and IL-21 variant. Provided herein are also methods of using the compositions described herein for the treatment of cancer.

### FIELD

The present disclosure relates generally to the field of molecular biology and medicine. More particularly, the methods and compositions herein are useful for treating cancer.

### BACKGROUND

Conventional cancer therapies generally target the proliferation, survival, or metabolic activity of tumor cells directly. In contrast, newly emerging cancer immunotherapies seek to restore anticancer immunity by modulating the tumor microenvironment (TME), tipping the equilibrium between factors that stimulate or inhibit anticancer immunity. Modern cancer immunotherapies, including immune checkpoint blockade, adoptive cell transfer, and cancer vaccines are ultimately dependent on immune cells (especially T cells) for their antitumor effects. While dramatic, durable and therapeutic responses are observed after checkpoint inhibitor therapy or CAR-T cell therapy, these successes are still limited to a small percentage of solid tumors, owing to the fact that the majority of solid tumors are characterized by a paucity of intratumoral T cell infiltrate, and defined as non-T cell-inflamed or “cold” tumors.

Therefore, new approaches that can improve intratumoral T cell infiltrate and transform “cold” tumors into “hot” or T cell-inflamed tumors are urgently needed to improve the efficacy of cancer immunotherapy.

### SUMMARY

Provided herein are IL-7/IL-21 fusion proteins comprising (i) IL-7 or an IL-7 variant and (ii) IL-21 or an IL-21 variant. Also described are vectors and immune cells for the delivery of fusion proteins comprising (i) IL-7 or an IL-7 variant and (ii) IL-21 or an IL-21 variant to a cancer cell, as well as methods of using the described fusion proteins, vectors, and immune cells for the treatment of cancer.

Provided herein are fusion proteins comprising (1) IL-7 or an IL-7 variant and (ii) IL-21 or an IL-21 variant.

In one embodiment, the IL-7 is human or murine IL-7. In some embodiments, the IL-7 or IL-7 variant comprises a sequence that is at least 90% identical to SEQ ID NO:14 or SEQ ID NO22. In some embodiments, the IL-7 comprises sequence SEQ ID ID NO: 14 or SEQ ID ID NO:22. In one embodiment, the IL-7 comprises sequence SEQ ID ID NO:22.

In some embodiments, the IL-21 is human or murine IL-21. In some embodiments, the IL-21 or IL-21 variant comprises a sequence that is at least 90% identical to SEQ ID ID NO: 12 or SEQ ID ID NO:20. In some embodiments, the IL-21 comprises SEQ ID ID NO: 12 or SEQ ID ID NO:20. In one embodiment, the IL-21 comprises SEQ ID ID NO:20.

In some embodiments, the fusion protein comprises a signal sequence. In some embodiments, the signal sequence comprises a sequence that is at least 90% identical to a sequence selected from SEQ ID NOs: 32-35. In some embodiments, the signal sequence comprises a sequence selected from SEQ ID NOs: 32-35.

Provided herein are fusion proteins comprising (i) IL-7 or an IL-7 variant and (ii) IL-21 or an IL-21 variant, wherein the IL-7 or IL-7 variant is linked to the IL-21 or IL-21 variant via a linker. In some embodiments, the linker is a polypeptide linker. In some embodiments, the polypeptide linker is a flexible linker. In some embodiments, the linker predominantly comprises glycines and serines. In some embodiments, the linker comprises SEQ ID NO:27 (GGGGS) or one or more repeats of SEQ ID NO:27 (GGGGS). In some embodiments, the linker comprises a sequence selected from the group consisting of GGS, SEQ ID NO:28 (GGSGGGS), SEQ ID NO:29 (GGGSGGGGS), GGGGSGGGGSGGGGS (SEQ ID NO:30), and SEQ ID NO:36 (GGGGGSGGGGSGGGGSGGGGS). In some embodiments, the polypeptide linker is a rigid linker. In some embodiments, the linker predominantly comprises alanines. In some embodiments, the linker comprises a sequence that is at least 90% identical to any one of SEQ ID NO:31 (A(EA₃K)₄AAA), SEQ ID NO:37 (A(EA₃K)₁AAA), and SEQ ID NO:38 (A(EA₃K)₄AAA). In one embodiment, the linker comprises a sequence selected from the group consisting of SEQ ID NO:31 (A(EA₃K)₄AAA), SEQ ID NO:37 (A(EA₃K)₁AAA), and SEQ ID NO:38 (A(EA₃K)₅AAA).

Provided herein are fusion proteins comprising (i) IL-7 or an IL-7 variant and (ii) IL-21 or an IL-21 variant, wherein the IL-7 or IL-7 variant is located N-terminally of the IL-21 or IL-21 variant. Provided herein are fusion proteins comprising (i) IL-7 or an IL-7 variant and (ii) IL-21 or an IL-21 variant, wherein the IL-7 or IL-7 variant is located C-terminally of the IL-21 or IL-21 variant.

Provided herein is a fusion protein comprising a sequence that is at least 90% identical to a sequence selected from SEQ ID NOs: 15-18, 23-26, 39-46, or 52-59. In some embodiments, the fusion protein comprises a sequence that is at least 90% identical to a sequence selected from SEQ ID NOs: 23-26 or 52-59. In some embodiments, the fusion protein comprises a sequence selected from SEQ ID NOs: 15-18, 23-26, 39-46, or 52-59. In some embodiments, the fusion protein comprises a sequence selected from SEQ ID NOs: 23-26 or 52-59.

In some embodiments, the fusion protein comprises a sequence that is at least 90% identical to a sequence selected from SEQ ID NOs:47-50 or 60-63. In some embodiments, the fusion protein comprises a sequence that is at least 90% identical to a sequence selected from SEQ ID NOs: 60-63. In some embodiments, the fusion protein comprises a sequence selected from SEQ ID NOs:47-50 or 60-63. In some embodiments, the fusion protein comprises a sequence selected from SEQ ID NOs: 60-63.

In some embodiments, the fusion protein is conjugated to one or more of a cytotoxin, a fluorescent label and an imaging agent.

Provided herein is a nucleic acid encoding a fusion protein provided herein. In some embodiments, the nucleic acid is isolated. Provided herein is a cell comprising a nucleic acid encoding a fusion protein provided herein. In some embodiments, the cell is a bacterial cell, a yeast cell, an insect cell, or a mammalian cell. In some embodiments, the cell is an immune cell. In some embodiments, the immune cell is a lymphocyte, a dendritic cell, a natural killer cell, or a macrophage. In some embodiments, the cell is a chimeric antigen receptor (CAR) T lymphocyte, a CAR macrophage, a CAR NK, a T cell receptor (TCR) T lymphocyte, or a tumor infiltration lymphocyte.

Provided herein is a vector comprising a nucleic acid encoding a fusion protein provided herein. In some embodiments, the vector is a viral vector. In some embodiments, the vector comprises a nucleic acid encoding a payload. In some embodiments, the payload is a cytokine, a chemokine, a tumor antigen, or a checkpoint inhibitor. In some embodiments, the viral vector is derived from an oncolytic virus. In some embodiments, the oncolytic virus is a vaccinia virus. Provided herein is a cell comprising a vector disclosed herein.

Provided herein is a viral particle comprising a nucleic acid encoding a fusion protein provided herein. In some embodiments, the viral particle further comprises a nucleic acid encoding a payload. In some embodiments, the payload is a cytokine, a chemokine, a tumor antigen, or a checkpoint inhibitor. In some embodiments, the viral particle derived from is an oncolytic virus. In one embodiment, the oncolytic virus is a vaccinia virus.

In some embodiments, provided is a viral vector or a viral particle comprising a vaccinia virus genome, wherein the vaccinia virus genome has a deletion of one or more of the thymidine kinase (TK) gene, the vaccinia growth factor (VGF gene), and the A56R gene (coding for hemagglutinin). In some embodiments, the vaccinia virus genome has a deletion of one or more of the viral gene for A41L, A44L, A46R, A49, A52R, A53R, B5R, B8R, B13R (SPI-2), B15R, B18R, C3L (VCP), C6, C7L, CI2L, E3L, FIL, KIL, K3L, K7R, MIL, and N1L.

Provided herein is a method for treating cancer, the method comprising administering to a subject in need thereof an effective amount of a fusion protein or of a viral particle disclosed herein. In some embodiment, the cancer is melanoma, pancreatic cancer, thyroid cancer, lung cancer, colorectal cancer, squamous cancer, prostate cancer, breast cancer, bladder cancer, gastric cancer, sarcoma, mesothelioma, ovarian cancer, endometrial cancer, or cervical cancer.

Provided herein is a method for reducing tumor growth, the method comprising administering to a subject in need thereof an effective amount of a fusion protein or of a viral particle disclosed herein.

Provided herein is a method for reducing tumor metastasis, the method comprising administering to a subject in need thereof an effective amount of a fusion protein or of a viral particle disclosed herein.

Provided herein is a method for increasing cytokine production, the method comprising administering to a subject in need thereof an effective amount of a fusion protein or of a viral particle disclosed herein.

Provided herein is a method for increasing anti-tumor immunity, the method comprising administering to a subject in need thereof an effective amount of a fusion protein or of a viral particle disclosed herein.

Provided herein is a method for increasing infiltration of a tumor with immune cells, the method comprising administering to a subject in need thereof an effective amount of a fusion protein or of a viral particle disclosed herein.

Provided herein is a method for reducing T cell tolerance, the method comprising administering to a subject in need thereof an effective amount of a fusion protein or of a viral particle disclosed herein.

Provided herein is a method for enhancing T cell expansion, the method comprising administering to a subject in need thereof an effective amount of a fusion protein or of a viral particle disclosed herein.

Provided herein is a method for increasing the number of memory T cells, the method comprising administering to a subject in need thereof an effective amount of a fusion protein or of a viral particle disclosed herein.

In some embodiments, the methods disclosed herein further comprise administering to the subject an additional antineoplastic agent. In some embodiments, the antineoplastic agent is a checkpoint inhibitor, a CAR T lymphocyte, a CAR macrophage, a CAR NK, a TCR T lymphocyte, or a tumor infiltration lymphocyte.

Provided herein is a method of making a fusion protein disclosed herein, the method comprising:
(a) providing a cell expressing a fusion protein disclosed herein; and
(b) expressing the fusion protein in the cell; and
(c) optionally substantially purifying the fusion protein.

### BRIEF DESCRIPTION OF THE FIGURES

FIG. 1 provides a schematic diagram of viral vectors. The indicated vectors were generated by homologous recombination of related genes of interest into the tk locus of vaccinia viral genome of VSC20. The cDNA for the genes of interest were generated via PCR using the templates/primers listed in Table 2. TKR, thynidine kinase right arm; TKL, thynidine kinase left arm. See Table 1 for viral vector nomenclature.

FIG. 2 illustrates viral vector replication and cytokine expression. Tumor cell MC38-luc (3×10⁵ cells), B16 (2×10⁵ cells) or AB12-luc (3×10⁵ cells), were mock-infected or infected with vvDD or vvDD expressing indicated cytokines or fusion proteins, respectively, at an MOT of 1. The cell pellets were harvested to measure A34R, IL-21 or IL-7 expression 24 hours after infection using RT-qPCR. Cytokine expression is presented relative to hypoxanthine phosphoribosyl transferase 1 (HPRT1) expression. HPRT1 served as a housekeeping gene. Vaccinia virus outer envelope protein A34 served as a control. Data represent two independent experiments. See Table 1 for viral vector nomenclature.

FIGS. 3A and 3B illustrate that a vvDD vector expressing fusion protein IL-21F7 or IL-21R7, respectively, elicits potent therapeutic effects in a murine colon cancer model. C57BL/6 mice were i.p. inoculated with 5×10⁵MC38-luc cells (FIG. 3A) or 3.5×10⁶ID8-luc cells (FIG. 3B) and treated with PBS, vvDD, or vvDD expressing the indicated cytokines at 2×10⁸ PFU/mouse nine days after tumor inoculation. The Kaplan Meier survival curve is shown. A log-rank (Mantel-Cox) test was used to compare survival rates. *P<0.05; **P<0.01; ***P<0.001; and ****P<0.0001. ns: not significant. See Table 1 for viral vector nomenclature.

FIGS. 4A and 4B illustrate that a vvDD vector expressing fusion protein IL-21F7 or IL-21R7, respectively, elicits potent tumor-specific antitumor effects at memory phase. MC38-luc-intraperitoneal-bearing C57BL/6 mice which were treated with vvDD expressing the indicated cytokine and survived more than 150 days, were s.c. injected with 1×10⁶ MC38 cells in the left flanks and 3×10⁵ B16 cells in the right flanks, respectively. A two-way ANOVA test was used to compare tumor growth curves for both the MC38 tumors (FIG. 4A) and the control B16 tumors (FIG. 4B). ****P<0.0001. See Table 1 for viral vector nomenclature.

FIG. 5 illustrates that the antitumor effected elicited by vvDD-IL-21R7 is dependent on CD8⁺ T cells, CD117⁺ cells (mast cell) and IFN-γ. B6 mice were i.p. inoculated with 5×10⁵ MC38-luc and treated with PBS or vvDD-IL-21R7 at 2×10⁸PFU/mouse nine days after tumor inoculation. Some of the vvDD-IL-21R7-treated groups were treated as follows: (1) α-CD8 antibodies (150 μg/injection) were injected at Day 5, 7, 9 and 11 after viral treatment, (2) α-CD4 antibodies (150 μg/injection) were injected at Day 5, 7, 9 and 11 after viral treatment, (3) α-IFN-γ antibodies (200 μg/injection) were injected at Day 5, 7, 9 and 11 after viral treatment, or (4) α-CD117 antibodies (200 μg/injection) were injected at Day 9, 11, 13 and 14 after tumor cell injection, respectively. See Table 1 for viral vector nomenclature.

FIG. 6. illustrates that virally-delivered fusion cytokines with different linkers elicited potent antitumor effects in a late-stage tumor model. B6 mice were inoculated i.p. with 5×10⁵ MC38-luc cells and treated with PBS, vvDD-IL-21+7, or vvDD-armed with fusion cytokines with different linkers, respectively, at 2×10⁸ PFU/mouse nine days after tumor inoculation. See Table 1 for viral vector nomenclature.

FIG. 7. illustrates that membrane-bound fusion cytokines elicited potent antitumor effects in a late-stage tumor model. B6 mice were inoculated i.p. with 5×10⁵ MC38-luc cells and treated with PBS, vvDD-IL-21R7, vvDD-IL-21R7-FG, or vvDD-IL-21R7-RG, respectively, at 2×10⁸ PFU/mouse nine days after tumor inoculation. See Table 1 for viral vector nomenclature.

### DETAILED DESCRIPTION

Provided herein are fusion proteins comprising (i) IL-7 or an IL-7 variant and (ii) IL-21 or an IL-21 variant. Also described are vectors and immune cells for the delivery of fusion proteins comprising (i) IL-7 or an IL-7 variant and (ii) IL-21 or an IL-21 variant to a cancer cell, as well as method of using the described fusion proteins, vectors, and immune cells for the treatment of cancer.

### Fusion Proteins

Provided herein are fusion proteins comprising (i) IL-7 or an IL-7 variant and (ii) IL-21 or an IL-21 variant, herein referred to as “IL-7/IL-21 fusion proteins” or “IL-21/IL-7 fusion proteins.”

As used herein, the term “IL-7 variant” refers to a modified IL-7 protein that comprises one or more alterations when compared to the parental protein, including, but not limited to amino acid additions, substitutions, insertions, deletions, or posttranslational modifications, wherein the IL-7 variant retains at least 10% of the immune-activating activity of the parental protein. A substituted or inserted amino acid residue may or may not be one encoded by the genetic code. The IL-7 variant may be a portion of the parental protein that comprises an immune-activating portion of the parental protein. In some embodiments, the IL-7 variant comprises one or more conservative mutations as compared to its parental counterpart. In some embodiments, the IL-7 variant comprises 1, 2, 3, 4, 5, 6, 7, 8, 9, or 10 conservative mutations as compared to its parental counterpart.

As used herein, the terms “conservative amino acid substitutions” and “conservative modifications” refer to amino acid modifications that do not significantly affect or alter the function and/or activity of the presently disclosed proteins comprising the amino acid sequence. Such conservative modifications include amino acid substitutions, additions, and deletions. Modifications can be introduced into the proteins of this disclosure by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine.

As used herein, the term “IL-21 variant” refers to a modified IL-21 protein that comprises one or more alterations when compared to the parental protein, including, but not limited to amino acid additions, substitutions, insertions, deletions, or posttranslational modifications, wherein the IL-21 variant retains at least 10% of the immune-activating activity of the parental protein. A substituted or inserted amino acid residue may or may not be one encoded by the genetic code. The IL-21 variant may be a portion of the parental protein that comprises an immune-activating portion of the parental protein. In some embodiments, the IL-21 variant comprises one or more conservative mutations as compared to its parental counterpart. In some embodiments, the IL-21 variant comprises 1, 2, 3, 4, 5, 6, 7, 8, 9, or 10 conservative mutations as compared to its parental counterpart.

In some embodiments, the IL-7 is a mammalian or a primate IL-7. In some embodiments, the IL-7 is a human, mouse, rat, hamster, rabbit, rodent, cow, pig, sheep, horse, goat, dog, or cat IL-7. In some embodiments, the IL-7 is human IL-7. In some embodiments, the IL-7 comprises a sequence that is at least 80%, at least 85%, at least 90%, at least 95%, at least 96%, at least 97%, at least 98%, or at least 99% identical to SEQ ID ID NO:22. In one embodiment, the IL-7 comprises sequence SEQ ID ID NO:22. In some embodiments, the IL-7 comprises a sequence that is at least 80%, at least 85%, at least 90%, at least 95%, at least 96%, at least 97%, at least 98%, or at least 99% identical to SEQ ID ID NO: 14. In one embodiment, the IL-7 comprises sequence SEQ ID ID NO:14. In some embodiments, the IL-7 is joined to a signal sequence, wherein the IL-7 with signal sequence comprises a sequence that is at least 80%, at least 85%, at least 90%, at least 95%, at least 96%, at least 97%, at least 98%, or at least 99% identical to SEQ ID ID NO:21. In one embodiment, the IL-7 with signal sequence comprises sequence SEQ ID ID NO:21. In some embodiments, the IL-7 is joined to a signal sequence, wherein the IL-7 with signal sequence comprises a sequence that is at least 80%, at least 85%, at least 90%, at least 95%, at least 96%, at least 97%, at least 98%, or at least 99% identical to SEQ ID ID NO: 13. In one embodiment, the IL-7 with signal sequence comprises sequence SEQ ID ID NO: 13.

In some embodiments, the IL-21 is a mammalian or a primate IL-21. In some embodiments, the IL-21 is a human, mouse, rat, hamster, rabbit, rodent, cow, pig, sheep, horse, goat, dog, or cat IL-21. In some embodiments, the IL-21 is human IL-21. In some embodiments, the IL-21 comprises a sequence that is at least 80%, at least 85%, at least 90%, at least 95%, at least 96%, at least 97%, at least 98%, or at least 99% identical to SEQ ID ID NO:20. In one embodiment, the IL-21 comprises sequence SEQ ID ID NO:20. In some embodiments, the IL-21 comprises a sequence that is at least 80%, at least 85%, at least 90%, at least 95%, at least 96%, at least 97%, at least 98%, or at least 99% identical to SEQ ID ID NO:12. In one embodiment, the IL-21 comprises sequence SEQ ID ID NO: 12. In some embodiments, the IL-21 is joined to a signal sequence, wherein the IL-21 with signal sequence comprises a sequence that is at least 80%, at least 85%, at least 90%, at least 95%, at least 96%, at least 97%, at least 98%, or at least 99% identical to SEQ ID ID NO: 19. In one embodiment, the IL-21 with signal sequence comprises sequence SEQ ID ID NO: 19. In some embodiments, the IL-21 is joined to a signal sequence, wherein the IL-21 with signal sequence comprises a sequence that is at least 80%, at least 85%, at least 90%, at least 95%, at least 96%, at least 97%, at least 98%, or at least 99% identical to SEQ ID ID NO:11 In one embodiment, the IL-21 with signal sequence comprises sequence SEQ ID ID NO: 11.

In some embodiments, the IL-7/IL-21 fusion protein comprises (i) a sequence that is at least 80%, at least 85%, at least 90%, at least 95%, at least 96%, at least 97%, at least 98%, or at least 99% identical to SEQ ID ID NO:22 and (ii) a sequence that is at least 80%, at least 85%, at least 90%, at least 95%, at least 96%, at least 97%, at least 98%, or at least 99% identical to SEQ ID ID NO:20. In some embodiments, the IL-7/IL-21 fusion protein comprises (i) a sequence comprising SEQ ID ID NO:22 and (ii) a sequence comprising SEQ ID ID NO:20.

In some embodiments, the IL-7/IL21 fusion protein comprises:
(a) (i) a sequence that is at least 80%, at least 85%, at least 90%, at least 95%, at least 96%, at least 97%, at least 98%, or at least 99% identical to SEQ ID ID NO:21 and (ii) a sequence that is at least 80%, at least 85%, at least 90%, at least 95%, at least 96%, at least 97%, at least 98%, or at least 99% identical to SEQ ID ID NO:20;
(b) (i) a sequence that is at least 80%, at least 85%, at least 90%, at least 95%, at least 96%, at least 97%, at least 98%, or at least 99% identical to SEQ ID ID NO:22 and (ii) a sequence that is at least 80%, at least 85%, at least 90%, at least 95%, at least 96%, at least 97%, at least 98%, or at least 99% identical to SEQ ID ID NO:19;
(c) (i) a sequence comprising SEQ ID ID NO:21 and (ii) a sequence comprising SEQ ID ID NO:20; or
(d) (i) a sequence comprising SEQ ID ID NO:22 and (ii) a sequence comprising SEQ ID ID NO: 19;

In some embodiments, the IL-7/IL-21 fusion protein comprises (i) a sequence that is at least 80%, at least 85%, at least 90%, at least 95%, at least 96%, at least 97%, at least 98%, or at least 99% identical to SEQ ID ID NO: 14 and (ii) a sequence that is at least 80%, at least 85%, at least 90%, at least 95%, at least 96%, at least 97%, at least 98%, or at least 99% identical to SEQ ID ID NO:12. In some embodiments, the IL-7/IL-21 fusion protein comprises (i) a sequence comprising SEQ ID ID NO:1 and (ii) a sequence comprising SEQ ID ID NO:2.

In some embodiments, the IL-7/IL-21 fusion protein comprises:
(a) (i) a sequence that is at least 80%, at least 85%, at least 90%, at least 95%, at least 96%, at least 97%, at least 98%, or at least 99% identical to SEQ ID ID NO: 13 and (ii) a sequence that is at least 80%, at least 85%, at least 90%, at least 95%, at least 96%, at least 97%, at least 98%, or at least 99% identical to SEQ ID ID NO:12;
(b) (i) a sequence that is at least 80%, at least 85%, at least 90%, at least 95%, at least 96%, at least 97%, at least 98%, or at least 99% identical to SEQ ID ID NO: 14 and (ii) a sequence that is at least 80%, at least 85%, at least 90%, at least 95%, at least 96%, at least 97%, at least 98%, or at least 99% identical to SEQ ID ID NO:11;
(c) (i) a sequence comprising SEQ ID ID NO:13 and (ii) a sequence comprising SEQ ID ID NO:12; or
(d) (i) a sequence comprising SEQ ID ID NO:14 and (ii) a sequence comprising SEQ ID ID NO:11;

Provided is an IL-7/IL-21 fusion protein in which the IL-7 protein or IL-7 variant is located N-terminally of the IL-21 protein or IL-21 variant. Provided is an IL-7/IL-21 fusion protein in which the IL-7 protein or IL-7 variant is located C-terminally of the IL-21 protein or IL-21 variant.

Provided are IL-7/IL-21 fusion proteins comprising one or more IL-7 proteins or IL-7 variants and/or one or more IL-21 proteins or IL-21 variants.

In some embodiments, the IL-7 protein or IL-7 variant is fused to a signal peptide. The terms “signal peptide” and “signal sequence” are used interchangeably herein. In some embodiments, the IL-21 protein or IL-21 variant is fused to a signal peptide. Sequences of suitable signal peptides are known in the art. Non-limiting example signal peptides include, but are not limited to, the first 22 amino acids of SEQ ID NO: 11 (murine IL-21 signal sequence), the first 25 amino acids of SEQ ID NO:13 (murine IL-7 signal sequence), the first 29 amino acids of SEQ ID NO: 19 (human IL-21 signal sequence), and the first 25 amino acids of SEQ ID NO:21 (human IL-7 signal sequence). In some embodiments, provided is an IL-7/IL-21 fusion protein comprising a signal sequence that is that is at least 80%, at least 85%, at least 90%, at least 95%, at least 96%, at least 97%, at least 98%, or at least 99% identical to any one of SEQ ID NOs: 32-35. In some embodiments, provided is an IL-7/IL-21 fusion protein comprising a signal sequence that comprises any one of SEQ ID NOs: 32-35.

In some embodiments, provided is an IL-7/IL-21 fusion protein comprising a sequence that is at least 80%, at least 85%, at least 90%, at least 95%, at least 96%, at least 97%, at least 98%, or at least 99% identical to any one of SEQ ID NOs:15-18, 23-26, 39-46, or 52-59. In some embodiments, provided is an IL-7/IL-21 comprising any one of SEQ ID NOs:15-18, 23-26, 39-46, or 52-59.

In some embodiments, provided is a membrane-bound IL-7/IL-21 fusion protein comprising a sequence that is at least 80%, at least 85%, at least 90%, at least 95%, at least 96%, at least 97%, at least 98%, or at least 99% identical to any one of SEQ ID NOs:47-50 or 60-63. In some embodiment, the membrane-bound IL-7/IL-21fusion protein comprises any one of SEQ ID NOs:47-50 or 60-63.

In some embodiments, provided is an IL-7/IL-21 fusion protein wherein one or more of amino acids 147-149 (i.e., the sequence GPA) of SEQ ID NOs: 15, 17, 39, 41, 43, 45 are replaced by other amino acids or deleted. In some embodiments, provided is an IL-7/IL-21 fusion protein wherein one or more of amino acids 163-165 (i.e., the sequence GPA) of SEQ ID NOs: 23, 25, 52, 54, 56, 58 are replaced by other amino acids or deleted.

### Linkers

In some embodiments, the IL-7 protein or IL-7 variant is directly fused to the IL-21 protein or IL-21 variant. In some embodiments, the IL-7 protein or IL-7 variant is covalently linked to the IL-21 protein or IL-21 variant through a linker.

In one embodiment, the linker is a flexible linker. In one embodiment, the linker is a rigid linker.

In one embodiment, the linker is a cleavable linker. In one embodiment, the linker is a non-cleavable linker.

In one embodiment, the linker is a helical linker. In one embodiment, the linker is a non-helical linker.

In some embodiments, the linker is a polypeptide linker. In some embodiments, the flexible linker is between 3 and 30 amino acids long.

In some embodiments, the flexible linker predominantly comprises glycines and serines. In some embodiments, the flexible linker comprises a sequence that is at least 80%, at least 85%, at least 90%, at least 95%, at least 96%, at least 97%, at least 98%, or at least 99% identical to one of the following sequences: GGS, SEQ ID NO:27 (GGGGS), SEQ ID NO:28 (GGSGGGS), SEQ ID NO:29 (GGGGSGGGGS), GGGGSGGGGSGGGGS (SEQ ID NO:30), and SEQ ID NO:36 (GGGGSGGGGSGGGGSGGGGS).

In embodiments, the linker comprises GGS, SEQ ID NO:27 (GGGGS), SEQ ID NO:28 (GGSGGGS), SEQ ID NO:29 (GGGGSGGGGS), GGGGSGGGGSGGGGS (SEQ ID NO:30), and SEQ ID NO:36 (GGGGSGGGGSGGGGSGGGS). In embodiments, the flexible linker comprises repeats of one or more flexible linkers disclosed herein.

In some embodiments, the rigid linker predominantly comprises alanines. In some embodiments, the rigid linker comprises a sequence that is at least 80%, at least 85%, at least 90%, at least 95%, at least 96%, at least 97%, at least 98%, or at least 99% identical to one of the following sequences: SEQ ID NO:31 (A(EA₃K)₄AAA), SEQ ID NO:37 (A(EA₃K)₁AAA), or SEQ ID NO:38 (A(EA₃K)₅AAA). In some embodiments, the rigid linker comprises SEQ ID NO:31 (A(EA₃K)₄AAA), SEQ ID NO:37 (A(EA₃K)₁AAA), or SEQ ID NO:38 (A(EA₃K)₅AAA). In embodiments, the rigid linker comprises repeats of one or more rigid linkers disclosed herein.

In embodiments, provided is a linker that comprises flexible and rigid portions.

Fusions with Functional Moieties

In some embodiments, the IL-7/IL-21 fusion protein is conjugated to a functional moiety, including, but not limited to, a diagnostic moiety, a detectable moiety, a therapeutic moiety, or a moiety useful for purification.

Examples of moieties useful for purification include, but are not limited to, Albumin-binding protein (ABP), Alkaline Phosphatase (AP), AUI epitope, AU5 epitope, Bacteriophage T7 epitope (T7-tag), Bacteriophage V5 epitope (V5-tag), Biotin-carboxy carrier protein (BCCP), Bluetongue virus tag (B-tag), Calmodulin binding peptide (CBP), Chloramphenicol Acetyl Transferase (CAT). Cellulose binding domain (CBP), Chitin binding domain (CBD), Choline-binding domain (CBD), Dihydrofolate reductase (DHFR), E2 epitope, FLAG epitope, Galactose-binding protein (GBP), Green fluorescent protein (GFP), (Glu-Glu (EE-tag), Glutathione S-transferase (GST), Human influenza hemagglutinin (HA), HaloTag®, Histidine affinity tag (HAT), Horseradish Peroxidase (HRP), HSV epitope, Ketosteroid isomerase (KSI), KT3 epitope, LacZ, Luciferase, Maltose-binding protein (MBP), Myc epitope, NusA, PDZ domain, PDZ ligand, Polyarginine (Arg-tag), Polyaspartate (Asp-tag), Polycysteine (Cys-tag), Polyhistidine (His-tag), Polyphenylalanine (Phe-tag), Profinity eXact, Protein C, S1-tag, S-tag, Streptavadin-binding peptide (SBP), Staphylococcal protein A (Protein A), Staphylococcal protein G(Protein G), Strep-tag, Streptavadin, Small Ubiquitin-like Modifier (SUMO), Tandem Affinity Purification (TAP), T7 epitope, Thioredoxin (Trx), TrpE, Ubiquitin, Universal, and VSV-G.

Examples of detectable moieties include, but are not limited to, fluorescent moieties or labels, imaging agents, radioisotopic moieties, radiopaque moieties, and the like, e.g., detectable labels such as biotin, fluorophores, chromophores, spin resonance probes, or radiolabels. Non-limiting examples of fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified protein that undergo structural changes upon binding a substrate (e.g., dansyl probes). Non-limiting examples of radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like). Other useful moieties are known in the art.

Examples of diagnostic moieties include, but are not limited to, detectable moieties suitable for revealing the presence of a disease or disorder. Typically, a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties include, but are not limited to, anti-inflammatory agents, anti-cancer agents, anti-neurodegenerative agents, anti-infective agents, or generally a therapeutic. The functional moiety may also have one or more of the herein-mentioned functions.

Non-limiting examples of therapeutic moieties include radionuclides with high-energy ionizing radiation that are capable of causing multiple strand breaks in nuclear DNA, and therefore suitable for inducing cell death (e.g., of a cancer cell). Non-limiting examples of high-energy radionuclides include: ⁹⁰Y, ¹²⁵I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high-energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Non-limiting examples of therapeutic moieties also include cytotoxic agents such as cytostatics (e.g., alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Non-limiting examples of therapeutic moieties also include alkylating agents such as the anthracycline family of drugs (e.g., adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Non-limiting examples of DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Non-limiting examples of therapeutic moieties also include transcription regulators such as actinomycin D, daunorubicin, doxorubicin, homoharringtonine, and idarubicin. Other non-limiting examples of cytostatic agents that are compatible with the embodiments disclosed herein include ansamycin benzoquinones, quinonoid derivatives (e.g., quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g., carmustine, lomustine, semustine).

Non-limiting examples of therapeutic moieties also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g., paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g., vinblastine, vincristine, vindesine, and vinorelbine (navelbine)); anti-angiogenesis compounds such as Angiostatin K1-3, DL-α-difluoromethyl-ornithine, endostatin, fumagillin, genistein, minocycline, staurosporine, and (±)-thalidomide.

Non-limiting examples of therapeutic moieties also include hormones and hormone antagonists, such as corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Non-limiting examples of therapeutic moieties also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Non-limiting examples of therapeutic moieties also include gene regulators such as 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Non-limiting examples of therapeutic moieties also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other cytotoxins that are compatible with the teachings herein include auristatins (e.g., auristatin E and monomethylauristan E), calicheamicin, gramicidin D, maytansanoids (e.g., maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

A moiety may have more than one function.

### Nucleic Acids, Genomes, and Vectors

Also provided herein are nucleic acids, genomes, and vectors comprising nucleic acids encoding the IL-7/IL-21 fusion proteins disclosed herein. The term “nucleic acid” as used herein refers to a polymeric form of nucleotides of any length, either ribonucleotides or desoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

Provided herein are nucleic acids comprising (i) a promoter and (ii) a transgene encoding an IL-7/IL-21 fusion protein disclosed herein, wherein the transgene is operably linked to the promoter. As used herein, “operably linked” refers to both expression control sequences that are contiguous with the transgene and expression control sequences that act in trans or at a distance to control the expression of the transgene. Expression control sequences include appropriate transcription initiation, termination, promoter and enhancer sequences; efficient RNA processing signals such as splicing and polyadenylation signals, sequences that stabilize cytoplasmic mRNA; sequences that enhance translation efficiency (e.g.,, Kozak consensus sequence); sequences that enhance protein stability; and when desired, sequences that enhance protein processing and/or secretion.

In some embodiments, the promoter is a viral promoter. In some embodiments, the promoter is a vaccinia virus promoter. Non-limiting examples of vaccinia virus promoters include p_{7.5}(E/L), p_{syn} (L), psₑ (L), or p_{H5} (E/L) (E/L=early and late promoter; L=late promoter). In one embodiment, the transgene encoding the IL-7/IL-21 fusion protein is operably linked to a p_{7.5} promoter.

In some embodiments, the nucleic acid comprises (i) a promoter, (ii) a transgene encoding an IL-7/IL-21 fusion protein disclosed herein, wherein the transgene is operatively linked to the promoter, and (iii) an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell.

In some embodiments, the nucleic acid comprises (i) a promoter, (ii) a transgene encoding an IL-7/IL-21 fusion protein disclosed herein, wherein the transgene is operatively linked to the promoter, and two tk gene flanking regions.

Provided herein is an oncolytic virus genome comprising (i) a promoter and (ii) a transgene encoding an IL-7/IL-21 fusion protein disclosed herein, wherein the transgene is operably linked to the promoter. Provided herein is an oncolytic virus genome comprising (i) a promoter, (ii) a transgene encoding an IL-7/IL-21 fusion protein disclosed herein, wherein the transgene is operatively linked to the promoter, and (iii) an internal ribosome entry site (IRES). In some embodiments, the promoter is a vaccinia virus p_{7.5} promoter.

Provided herein is an oncolytic virus genome having a deletion of or an inactivating mutation in one or more of the following non-limiting examples of genes: ribonucleotide reductase-large subunit, ribonucleotide reductase-small subunit, DNA ligase, dUTPase, tk, vgf, or A56R gene. Provided herein is an oncolytic virus genome having a deletion of or an inactivating mutation in one or more of the viral genes for A41L, A44L, A46R, A49, A52R, A53R, B5R, B8R, B13R (SPI-2), BI15R, B18R, C3L (VCP), C6, C7L, C12L, E3L, FIL, KIL, K3L, K7R, M1L, and N1L.

In some embodiments, the oncolytic virus genome is a vaccinia virus genome.

Provided herein is a vector comprising a nucleic acid comprising (i) a promoter and (ii) a transgene encoding an IL-7/IL-21 fusion protein disclosed herein, wherein the transgene is operably linked to the promoter. Provided herein is a vector comprising a nucleic acid comprising (i) a promoter, (ii) a transgene encoding an IL-7/IL-21 fusion protein disclosed herein, wherein the transgene is operatively linked to the promoter, and (iii) an internal ribosome entry site (IRES). In some embodiments, the promoter is a vaccinia virus p_{7.5} promoter. “Vector,” as used herein, means a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus derived sequences). A vector may also refer to a viral particle comprising a nucleic acid to be delivered into a host cell, either in vitro or in vivo.

In some embodiments, the vector comprises a nucleic acid encoding a payload. In some embodiments, the payload is, for example, a cytokine, a chemokine, a tumor antigen, or a checkpoint inhibitor. In some embodiments, the checkpoint inhibitor is an anti-PD1/L1 minibody.

### Anchoring Peptides

Provided herein are IL-7/IL-21 fusion proteins that are linked to an anchoring peptide, nucleic acids encoding an IL-7/IL-21 fusion protein that is linked to an anchoring peptide, oncolytic viruses comprising a nucleic acid encoding an IL-7/IL-21 fusion protein that is linked to an anchoring peptide, and cells expressing an IL-7/IL-21 fusion protein that is linked to an anchoring peptide.

In certain embodiments, an anchoring peptide is any protein which, when fused to an IL-7/IL-21 fusion protein, anchors the immunomodulator molecule to a cell membrane. In certain embodiments, an anchoring peptide can be used to modify an IL-7/IL-21 fusion protein to bind to the membrane of a cell (e.g., by adding a GPI or other molecule).

In some embodiments, the anchoring peptide is between about 10 and about 50 amino acids, or between about 15 and about 30, or about 20 amino acids in length.

In some embodiments, the anchoring peptide is a glycosylphosphatidylinositol (GPI)-anchor acceptor peptide. In some embodiments, the GI anchor acceptor peptide comprises at least a portion of the C terminus of its native peptide, for example comprises at least a portion of the 100 C-terminal amino acids, or at least a portion of the 50 C-terminal amino acids, of the native peptide, or at least a portion of the 30 C-terminal amino acids. In some embodiments, the anchoring peptide comprises a sequence that is at least is at least 80%, at least 85%, at least 90%, at least 95%, at least 96%, at least 97%, at least 98%, or at least 99% identical to SEQ ID NO:51. In one embodiment, the anchoring peptide comprises SEQ ID NO:51. In some embodiments, the fusion protein is linked to the anchoring peptide via a rigid linker, including, but not limited to, a rigid linker disclosed herein. In some embodiments, the fusion protein is linked to the anchoring peptide via a flexible linker, including, but not limited to, a flexible linker disclosed herein.

In some embodiments, the anchoring peptide can comprise sequences of various proteins as well as portions of cell membrane proteins that would serve essentially the same function. As exemplified by GPI, the anchoring function can be achieved by a modification of an underlying peptide or protein that contains non-peptide elements, including but not limited to carbohydrate, lipid, etc.

In some embodiments, the anchoring peptide can comprise a glycosylphosphatidylinositol (GPI)-anchor acceptor sequence of human CD16b anchor acceptor peptide. In some embodiments, the anchor is a GPI anchor acceptor peptide from a protein as set forth in Ferguson et al., “Chapter 11: Glycosylphosphatidyl Anchors” in Glycobiology 2nd Edition, Varki et al., editors, Cold Spring Harbor Press, 2009, the contents of which is incorporated in its entirety by reference herein, for example but not limited to alkaline phosphatase, CD58, CD14, NCAM-120 and TAG-1.

In some non-limiting embodiments, the GPI anchor acceptor peptide comprises a signal peptide portion (“SPP”), which functions during GPI addition and is cleaved in the process. In certain embodiments, a SPP comprises three domains: (1) a first domain comprising three relatively small amino acids (for example, but not limited to, Gly (G), Ala (A), Ser (S), Asn (N), Asp (D), or Cys (C) or any combination thereof), ω, (ω+1), and (ω+2), where ω is attached to the GPI anchor and (ω+1) and (ω+2) are the first 2 residues of the cleaved peptide; (2) a relatively polar domain spacer of about 5-10 amino acid residues and (3) a hydrophobic domain of about 15-20 amino acids. In certain embodiments, the anchoring peptide can further comprise a sequence that targets it to the endoplasmic reticulum to facilitate addition of the GPI anchor if that is not a feature viral infection (see Mayor S and Riezman H, 2004, Nature Reviews Molecular Cell Biology 5, 110-120, the contents of which is incorporated in its entirety by reference herein.) This signal peptide portion, extending from the protein C terminus, can be diagrammed as: ω−(ω+1)−(ω+2)-polar spacer region-hydrophobic domain (see Galian C et al., 2012, J. Biol. Chem. 287(20):16399-16409). In certain non-limiting embodiments, the ω−(ω+1)−(ω+2)-polar spacer region can include a sequence of about ten amino acids of which at least about 2 or at least about 3 residues are G and at least about 2 or at least about 3 or at least about 4 or at least about 5 residues are S. In some embodiments, the hydrophobic domain can include between about 15 and about 20 amino acids comprising at least about 10 residues selected from the group of A, Leu (L), Val (V), Phe (F) and combinations thereof.

### Oncolytic Viruses

In one aspect, the transgene encoding an IL-7/IL-21 fusion protein comprising IL-7 and IL-21 disclosed herein is delivered to a cancer cell using an oncolytic virus (OV). As such, provided herein are oncolytic viral vectors comprising a nucleic acid encoding an IL-7/IL-21 fusion protein disclosed herein. As used herein, an “oncolytic virus” is a virus that exhibits increased replication in, and lysis of, cancer cells relative to comparable non-cancer cells. In certain embodiments, the oncolytic virus exhibits selective replication in cancer cells and less or essentially no replication in non-cancer cells. In certain embodiments, less replication means that replication in cancer cells versus comparable non-cancer cells is at least about 30 percent greater, or at least about 50 percent greater, or at least about 80 percent greater. Non-limiting examples of oncolytic viruses include types of (i) adenovirus (“Ad”), for example hTERT-Ad; (ii) herpes simplex virus (“HSV”), for example G207, HSV-1716, T-VEC, and HSV-2 APK mutant; (iii) poxvirus, for example vaccinia virus, for example vSP and vvDD (tk″/vgf′); (iv) arbovirus; (v) paramyxovirus, for example, measles virus, mumps and Newcastle disease virus; (vi) rhabdovirus, for example, vesicular stomatitis virus; (vii) picornavirus, for example Coxsackie virus, Seneca Valley Virus, and polio virus; (viii) reovirus; (ix) parvovirus; and (x) recombinant/engineered versions of any of the oncolytic viruses disclosed herein.

OVs can directly kill tumor cells like conventional cancer therapies. This killing further provides a natural repertoire of tumor-specific antigens/tumor-associated antigens (TSAs/TAAs), danger signals (including damage-associated molecular pattern (DAMN/IP) and OV-derived pathogen-associated molecular pattern (PAMP) molecules) and inflammatory cytokines to trigger innate and adaptive anti-tumor immunity. This anti-tumor immune reactivity in turn results in the infiltration of diverse immune cells, including T lymphocytes, into the TME, transforming “cold” tumors. Oncolytic viruses have positive effects on almost every aspect of the cancer-immunity cycle and can be further armed with transgenes coding for proteins, including, but not limited to chemokines and cytokines, that can directly or indirectly help transform the T-ME so as to harness the immune system to attack and treat tumors.

In some embodiments, the oncolytic virus is an oncolytic virus that has been approved by the Food and Drug Administration (FDA) or is undergoing clinical trials. For example, but not by way of limitation, the oncolytic virus can be Talimogene laherparepvec (Imlygic™; Amgen, Inc.), also referred to as T-VEC. In certain embodiments, the oncolytic virus can be pelareorep (Reolysin®; Oncolytics Biotech, Inc.). In certain embodiments, the oncolytic virus can be DNX-2401 (DNAtrix Therapeutics). In certain embodiments, the oncolytic virus can be H101 (Oncorine®; Shanghai Sunway Biotech Co., Ltd.). In certain embodiments, the oncolytic virus can be pexastimogene devacirepvec (JX-594; SillaJen Inc.). In certain embodiments, the oncolytic virus can be CG0070 (Cold Genesys, Inc.). In certain embodiments, the oncolytic virus can be G47Δ (Daiichi-Sankyo Company, Limited).

In some embodiments, the oncolytic virus is a vaccinia virus. In certain non-limiting embodiments, the oncolytic virus is an engineered (also referred to as “recombinant”) vaccinia virus. In certain non-limiting embodiments, the virus is a recombinant vaccinia virus based on the Western Reserve (“WR”) strain of vaccinia, for example, the WR strain commercially available from the American Type Culture Collection as ATCC No. VR1354. Other vaccinia virus strains suitable for engineering include, but are not limited, to the Wyeth strain (ATCC VR-1536), the Lederle-Chorioallantoic strain (ATCC VR-325), and the CL strain (ATCC VR-117).

In some embodiments, the oncolytic virus is an engineered double-deleted vaccinia virus (vvDD) vaccinia viral construct comprising, for example, a modified version of a virus described in U.S. Pat. No. 8,506,947, McCart et al., 2001, Cancer Research 61:8751-8757, and/or Thorne S et al., 2007, J. Clin. Invest. 117:3350-3358, all of which are incorporated by reference herein in their entireties. For example, but not by way of limitation, the vaccinia virus can have deletions of the thymidine kinase (tk) and/or vaccinia growth factor (vgf) genes.

In some embodiments, a vaccinia virus has an inactivating mutation or deletion in one or more genes where the product of said gene or genes functions in viral replication. For example, but not by way of limitation, one or more of the following genes can bear an inactivating mutation or deletion: ribonucleotide reductase-large subunit, ribonucleotide reductase-small subunit, DNA ligase, dUTPase, tk, vgf, or A56R (coding for hemagglutinin) gene. In certain embodiments, an inactivating mutation is a mutation that either reduces or eliminates activity of the gene product. In certain embodiments, gene activation can be achieved by mutagenesis, e.g., site-directed mutagenesis or PCR-mediated mutagenesis. Alternatively, or additionally, in some embodiments, a nucleic acid can be inserted into one or more of the foregoing genes to achieve inactivation. In some embodiments, a nucleic acid encoding a protein can be inserted into one or more of the foregoing genes to achieve inactivation and to further achieve expression of the nucleic acid. In some embodiments, a nucleic acid encoding an immunomodulator molecule can be inserted within one of the foregoing genes to achieve inactivation.

In some embodiments, the oncolytic virus is a vaccinia virus having an inactivating mutation (a mutation that either reduces or eliminates activity of the gene product) in the tk gene. For example, but not limited to, the inactivation of the thymidine kinase gene can be generated by the insertion of a cytosine deaminase (fcy1) gene within the thymidine kinase gene locus of the vaccinia viral genome, resulting in the expression of the fcy1 gene rather than the tk gene. In another non-limiting embodiment, a nucleic acid encoding a detectable protein, for example a fluorescent protein, for example yellow fluorescent protein (“yfp”), can be inserted into the tk gene, thereby inactivating it. In certain embodiments, a nucleic acid encoding an immunomodulator can be inserted into the tk gene.

In additional or alternative embodiments, the recombinant vaccinia virus can have an inactivating mutation in the vaccinia growth factor gene. For example, but not by way of limitation, an insertion of a lacZ gene within the vgf gene locus results in the expression of the lacZ gene rather than vgf. In certain embodiments, a nucleic acid encoding an immunomodulator can be inserted into the vgf gene.

In some embodiments, the vaccinia virus is modified to reduce immunogenicity in a mammal, including, but not limited to in a human. In some embodiments, the vaccinia virus has a deletion of or an inactivating mutation in one or more of the viral genes for A41L, A44L, A46R, A49, A52R, A53R, B5R, B8R, BI3R (SPI-2), B15R, BI8R, C3L (VCP), C6, C7L, CI2L, E3L, F1L, KIL, K3L, K7R, MIL, and NIL.

### Construction of Oncolytic Viral Vectors

Methods for generating oncolytic viral vectors (including vaccinia virus vectors) that comprise transgenes are known in the art.

For example, a vaccinia viral particle comprising a nucleic acid comprising a transgene may be generated by transfecting a shuttle vector or plasmid containing transgene operatively linked to a vaccinia virus promoter into a cell that has been infected with vaccinia virus and introducing the exogenous sequence by homologous recombination. Alternative methods may be used, employing two viruses, one defective for some genes and one wild-type acting as a helper. In both cases, the recombinant viruses are produced by homologous recombination in the infected cell. Selection techniques may be employed to select the resulting, recombinant viral particles. In some embodiments, the promoter and the transgene operatively linked to the promoter are flanked by tk gene flanking sequences, so that they replace a portion or all of the genomic tk gene during the homologous recombination process.

In another variation, the bacteriophage T7 RNA polymerase gene can be integrated into the genome of vaccinia so that a transgene controlled by a T7 promoter, either in a transfected plasmid or a recombinant vaccinia virus, is expressed.

### Cells Expressing IL-7/IL-21 Fusion Proteins

In one aspect, provided is a cell comprising a transgene encoding an IL-7/IL-21 fusion protein disclosed herein. Provided is a method of making an IL-7/IL-21 fusion protein disclosed herein, the method comprising (i) providing a cell comprising a transgene encoding an IL-7/IL-21 fusion protein disclosed herein; and (ii) expressing the IL-7/IL-21 fusion protein in the cell. In some embodiments, the IL-7/IL-21 fusion protein is substantially purified from the cell. In some embodiments, provided is a cell comprising a transgene encoding an IL-7/IL-21 fusion protein disclosed herein, wherein the cell secretes the IL-7/IL-21 fusion protein. In some embodiments, the cell is a bacterial cell, a yeast cell, an insect cell, or a mammalian cell. In some embodiments, the cell is an immune cell.

In one aspect, the IL-7/IL-21 fusion protein is delivered to a cancer cell using an immune cell. In some embodiments, the IL-7/IL-21 fusion protein is expressed on the surface of the immune cell. In some embodiments, the IL-7/IL-21 fusion protein is fused to an anchoring peptide that anchors the fusion protein in the immune cell membrane.

In some embodiments, the immune cell further expresses on its surface a moiety that targets the immune cell to a cancer cell. In some embodiments, the targeting moiety recognizes a cancer antigen. Non-limiting examples of cancer antigen include CD19, CD20, CD30, CD33, CD38, CD133, BCMA, TEM8, EpCAM, ROR1, Folate Receptor, CD70, MAGE-1, MAGE-2, MAGE-3, MAGE A-10, MAGE-C2, MAGE-A12, CEA, tyrosinase, midkin, BAGE, CASP-8, β-catenin, CA-125, CDK-1, ESO-1, gp75, gp100, MART-1, MUC-1, MUM-1, p53, PAP, PSA, PSMA, ras, trp-1, HER-2, TRP-1, TRP-2, IL13Ralpha, IL13Ralpha2, AIM-2, AIM-3, NY-ESO-1, C9orf112, SART1, SART2, SART3, BRAP, RTN4, GLEA2, TNKS2, KIAA0376, ING4, HSPH1, C13orf24, RBPSUH, C6orf153, NKTR, NSEP1, U2AFIL, CYNL2, TPR GOLGA, BMI1, COX-2, EGFRvIII, EZH2, LICAM, Livin, Livinβ, MRP-3, Nestin, OLIG2, ART1, ART4, B-cycline, Gli1, Cav-1, Cathepsin B, CD74, E-Cadherin, EphA2/Eck, Fra-1/Fosl 1, GAGE-1, Ganglioside/GD2, GnT-V, β1, 6-N, Ki67, Ku70/80, PROXI, PSCA, SOX10, SOX1, Survivin, βhCG, WT1, mesothelin, melan-A, NY-BR-1, NY-CO-58, MN (gp250), telomerase, SSX-2, PRAME, PLK1, VEGF-A, VEGFR2, and Tie-2. In some embodiments, the targeting moiety recognizes a neoantigen. In some embodiments, the targeting moiety is a chimeric antigen receptor (CAR). In some embodiments, the targeting moiety is a T-cell receptor (TCR).

In some embodiments, the immune cell is a lymphocyte. In one embodiment, the immune cell is a T lymphocyte. In one embodiment, the immune cell is a B lymphocyte. In one embodiment, the immune cell is a natural killer cell. In one embodiment, the immune cell is a macrophage. In one embodiment, the immune cell is a dentritic cell. In some embodiments, the immune cell is a chimeric antigen receptor (CAR) T lymphocyte, a CAR macrophage, a CAR-NK, a T cell receptor T lymphocyte, or a tumor infiltration lymphocyte.

### Pharmaceutical Compositions

Provided herein are pharmaceutically acceptable compositions that comprise an IL-7/IL-21 fusion protein disclosed herein or a nucleic acid encoding an IL-7/IL-21 fusion protein disclosed herein formulated together with one or more pharmaceutically acceptable excipients. Also provided herein are pharmaceutically acceptable compositions that comprise an oncolytic virus comprising a nucleic acid encoding an IL-7/IL-21 fusion protein disclosed herein formulated together with one or more pharmaceutically acceptable excipients. Also provided herein are pharmaceutically acceptable compositions that comprise an immune cell expressing on its surface an IL-7/IL-21 fusion protein disclosed herein formulated together with one or more pharmaceutically acceptable excipients.

The active agent and excipient(s) may be formulated into compositions and dosage forms according to methods known in the art. The pharmaceutical compositions disclosed herein may be specially formulated in solid or liquid form, including those adapted for parenteral administration, for example, by subcutaneous, intratumoral, intramuscular or intravenous injection as, for example, a sterile solution or suspension.

Therapeutic compositions comprising an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline, pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present disclosure include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present disclosure include polysorbates (e.g., polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g., Pluronics, PF68, etc,).

Preservatives may be used in formulations disclosed herein. Suitable preservatives for use in the formulations disclosed herein include octadecyldimethylbenzyl ammonium chloride, hexamethonium chloride, benzalkonium chloride (a mixture of alkylbenzyl-dim-ethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

### Methods of Treatment

Provided herein is a method of treating cancer in a subject in need thereof, the method comprising administering to the subject in need thereof an IL-7/IL-21 fusion protein disclosed herein or a nucleic acid encoding an IL-7/IL-21 fusion protein disclosed herein. Provided herein is a method of treating cancer in a subject in need thereof, the method comprising administering to the subject an oncolytic virus comprising a nucleic acid encoding an IL-7/IL-21 fusion protein disclosed herein. Provided herein is a method of treating cancer in a subject in need thereof, the method comprising administering to the subject an immune cell expressing on its surface an IL-7/IL-21 fusion protein disclosed herein.

By “subject” is meant a mammal, including, but not limited to, a human or non-human mammal, such as a bovine, equine, canine, ovine, or feline, etc. Individuals and patients are also subjects herein.

“Therapeutically effective amount” means an amount of an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein that, when administered to a subject, is effective in producing the desired therapeutic effect. For example, the term “therapeutically effective” amount can refer to the amount of an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein that is sufficient to elicit the biological or medical response of a cell, tissue, system, animal or human.

The term “cancer” refers to or describes the physiological condition in mammals that is typically characterized by unregulated cell growth. Included in this definition are benign and malignant cancers, as well as dormant tumors or micrometastases. Accordingly, the term “cancer” as used herein refers to an uncontrolled growth of cells, which interferes with the normal functioning of the bodily organs and systems, including cancer stein cells and tumor vascular niches. A subject that has a cancer is a subject having objectively measurable cancer cells present in the subject's body. Included in this definition are benign and malignant cancers, as well as dormant tumors or micrometastases. Cancers that migrate from their original location and seed vital organs can eventually lead to the death of the subject through the functional deterioration of the affected organs.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms, diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder, or slow its course of development.

Provided herein are fusion proteins comprising IL-7 and IL-21 and nucleic acids encoding fusion proteins comprising IL-7 and IL-21 for use in the treatment of cancer. Provided herein is an oncolytic virus comprising a nucleic acid encoding an IL-7/IL-21 fusion protein disclosed herein for use in the treatment of cancer. Provided herein is an immune cell expressing on its surface an IL-7/IL-21 fusion protein disclosed herein for use in the treatment of cancer.

Provided herein is the use of an IL-7/IL-21 fusion protein disclosed herein or a nucleic acid encoding an IL-7/IL-21 fusion protein disclosed herein in the manufacture of a medicament for the treatment of cancer. Provided herein is the use of an oncolytic virus comprising a nucleic acid encoding an IL-7/IL-21 fusion protein disclosed herein in the manufacture of a medicament for the treatment of cancer. Provided herein is the use of an immune cell expressing on its surface an IL-7/IL-21 fusion protein disclosed herein in the manufacture of a medicament for the treatment of cancer.

Also provided herein is a method of reducing tumor growth in subject in need thereof, the method comprising administering to a subject an IL-7/IL-21 fusion protein disclosed herein or a nucleic acid encoding an IL-7/IL-21 fusion protein disclosed herein. Provided herein is a method of reducing tumor growth, the method comprising administering to a subject in need thereof an oncolytic virus comprising a nucleic acid encoding an IL-7/IL-21 fusion protein disclosed herein. Provided herein is a method of reducing tumor growth, the method comprising administering to a subject in need thereof an immune cell expressing on its surface an IL-7/IL-21 fusion protein disclosed herein. “Reducing” includes inhibiting and/or reversing and can refer to, for example, the symptoms of the disorder being treated, the presence or size of metastases or micrometastases, the size of the primary tumor, the presence or the size of the dormant tumor.

The embodiments disclosed herein may be used for treating metastasis, which relates to the spreading of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Accordingly, provided herein is a method of reducing metastasis in subject in need thereof, the method comprising administering to a subject an IL-7/IL-21 fusion protein disclosed herein or a nucleic acid encoding an IL-7/IL-21 fusion protein disclosed herein. Provided herein is a method of reducing metastasis, the method comprising administering to a subject in need thereof an oncolytic virus comprising a nucleic acid encoding an IL-7/IL-21 fusion protein disclosed herein. Provided herein is a method of reducing metastasis, the method comprising administering to a subject in need thereof an immune cell expressing on its surface an IL-7/IL-21 fusion protein disclosed herein.

Also contemplated are methods of reducing cancer stemness by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein. Cancer stemness may refer to the ability of a cell to self-renew and to generate an additional, phenotypically distinct cell type. Cancer stem cells (CSCs) are cancer cells that exhibit stem-cell like properties. CSCs often exhibit at least one hallmark of cancer, and are capable of generating at least one additional, phenotypically distinct cell type. Furthermore, cancer stem cells are capable of both asymmetric and symmetric replication. It is appreciated that a cancer stem cell may result from differentiated cancer cells that acquire stemness traits and/or stem cells that acquire phenotypes associated with cancer cells. Alternatively, cancer stem cells can reconstitute non-stromal cell types within a tumor.

Provided herein are methods of increasing cytokine production in the tumor microenvironment by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Provided herein are methods of increasing anti-tumor immunity in a subject by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Provided herein are methods of increasing infiltration of a tumor with immune cells by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Provided herein are methods of reducing T-cell tolerance by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Provided herein are methods of enhancing T cell expansion by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Provided herein are methods of increasing the number of memory T cells (or of increasing the ratio of memory T cell to non-memory T cells) by administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Also contemplated are methods of inducing cell death of a dividing cancer cell, the method comprising contacting the cancer cell with an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein.

Examples of cancer that can be treated with the compositions and methods disclosed herein include, but are not limited to, carcinoma, lymphoma, blastoma, and sarcoma. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; mesothelioma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; vulvar sarcomas; as well as other carcinomas and sarcomas.

The efficacy of the treatment methods for cancer comprising administering an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein can be measured by various endpoints commonly used in evaluating cancer treatments, including but not limited to, tumor regression, tumor weight or size shrinkage, time to progression, duration of survival, progression free survival, overall response rate, duration of response, and quality of life. The treatment with an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth, and/or relieve to some extent one or more of the symptoms associated with the disorder. In cases where a patient has more than one type of cancer, the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein can be effective in treating at least one of the cancers. To the extent the compositions disclosed herein act to prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

### Administration

The IL-7/IL-21 fusion proteins disclosed herein, the nucleic acids encoding an IL-7/IL-21 fusion protein disclosed herein, the oncolytic viruses comprising a nucleic acid encoding an IL-7/IL-21 fusion protein disclosed herein, or the immune cells expressing on their surface an IL-7/IL-21 fusion protein disclosed herein can be administered according to any known method in the art.

For example, but not by way of limitation, a method for the delivery of an IL-7/IL-21 fusion protein disclosed herein, a nucleic acid encoding an IL-7/IL-21 fusion protein disclosed herein, an oncolytic virus comprising a nucleic acid encoding an IL-7/IL-21 fusion protein disclosed herein, or an immune cell expressing on its surface an IL-7/IL-21 fusion protein disclosed herein or pharmaceutical compositions thereof can be via intratumoral injection. In certain embodiments, alternate methods of administration can also be used, e.g., intravenous, via infusion, parenteral, intravenous, intradermal, intramuscular, transdermal, rectal, intraurethral, intravaginal, intranasal, intrathecal, or intraperitoneal. The routes of administration can vary with the location and nature of the tumor. In certain embodiments, the route of administration can be intradental, transdermal, parenteral, intravenous, intramuscular, intranasal, subcutaneous, regional (e.g., in the proximity of a tumor, particularly with the vasculature or adjacent vasculature of a tumor), percutaneous, intrathecal, intratracheal, intraperitoneal, intraarterial, intravesical, intratumoral, inhalation, perfusion, by lavage or orally. In certain embodiments, the modified virus can be administered to the patient from a source implanted in the patient.

In certain embodiments, administration of the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can occur by continuous infusion over a selected period of time. In certain embodiments, the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be administered at a therapeutically effective dose by infusion over a period of about 15 minutes, about 30 minutes, about 45 minutes, about 50 minutes, about 55 minutes, about 60 minutes, about 75 minutes, about 90 minutes, about 100 minutes, or about 120 minutes or longer.

The IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be administered as a liquid dosage, wherein the total volume of administration is about 1 ml to about 5 ml, about 5 ml to 10 ml, about 15 ml to about 20 ml, about 25 ml to about 30 ml, about 30 ml to about 50 ml, about 50 ml to about 100 ml, about 100 ml to 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to 500 ml, about 500 ml to 750 ml or about 750 ml to 1000 ml.

In some embodiments, a single dose of the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can refer to the amount administered to a subject or a tumor over a 1, 2, 5, 10, 15, 20 or 24 hour period. In certain embodiments, the dose can be spread over time or by separate injection. In certain embodiments, multiple doses (e.g., 2, 3, 4, 5, 6 or more doses) of the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be administered to the subject, for example, where a second treatment can occur within 1, 2, 3, 4, 5, 6, 7 days or weeks of a first treatment. In certain embodiments, multiple doses of the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be administered to the subject over a period of 1, 2, 3, 4, 5, 6, 7 or more days or weeks. In certain embodiments, the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months).

An effective amount of the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be determined by methods known in the art. In certain embodiments, the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical compositions thereof can be administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor.

In certain embodiments, the amount of the oncolytic virus comprising a nucleic acid encoding a fusion protein disclosed herein or a pharmaceutical composition thereof administered can be between about 1×10⁷ and 1×10¹⁰ infectious viral particles or plaque forming units (pfu), or between about 1×10 and 1×10⁹ pfu/m² surface area of the subject to be treated. In certain embodiments, the oncolytic virus comprising a nucleic acid encoding the fusion protein cart be administered at a dose that can comprise about 1×10⁸ pfu. In certain embodiments, the amount of virus administered can be between about 1×10³ and 1×10¹² viral particles or pfu, or between about 1×10⁵ and 1×10¹⁰ pfu, or between about 1×10⁵ and 1×10⁸ pfu, or between about 1×10⁸ and 1×10¹⁰ pfu. In certain embodiments, the oncolytic virus comprising a nucleic acid encoding the fusion protein can be administered at a dose that can comprise about 1×10³ pfu/dose to about 1×10⁴ pfu/dose, about 1×10⁴ pfu/dose to about 1×10⁵ pfu/dose, about 1×10⁵ pfu/dose to about 1×10⁶ pfu/dose, about 1×10⁷ pfu/dose to about 1×10⁵ pfu/dose, about 1×10⁹ pfu/dose to about 1×10¹⁰ pfu/dose, about 1×10¹⁰ pfu/dose to about 1×10¹¹ pfu/dose, about 1×10¹¹ pfu/dose to about 1×10¹² pfu/dose, about 1×10¹² pfu/dose to about 1×10¹³ pfu/dose, about 1×10¹³ pfu/dose to about 1×10¹⁴ pfu/dose, or about 1×10¹⁴ pfu/dose to about 1×10¹⁵ pfu/dose. In certain embodiments, the oncolytic virus comprising a nucleic acid encoding the fusion protein can be administered at a dose that can comprise about 1×10³ viral particles/dose to about 1×10⁴ viral particles/dose, about 1×10⁴ viral particles/dose to about 1×10⁵ viral particles/dose, about 1×10⁵ viral particles/dose to about 1×10⁶ viral particles/dose, about 1×10⁷ viral particles/dose to about 1×10⁸ viral particles/dose, about 1×10⁹ viral particles/dose to about 1×10¹⁰ viral particles/dose, about 1×10¹⁰ viral particles/dose to about 1×10¹¹ viral particles/dose, about 1×10¹³ viral particles/dose to about 1×10¹² viral particles/dose, about 1×10¹² viral particles/dose to about 1×10¹³ viral particles/dose, about 1×10¹³ viral particles/dose to about 1×10¹⁴ viral particles/dose, or about 1×10¹⁴ viral particles/dose to about 1×10¹⁵ viral particles/dose.

### Combination Therapies

In one aspect, provided herein is an IL-7/IL-21 fusion protein disclosed herein, a nucleic acid encoding an IL-7/IL-21 fusion protein disclosed herein, a oncolytic virus comprising a nucleic acid encoding an IL-7/IL-21 fusion protein disclosed herein, or an immune cell expressing on its surface an IL-7/IL-21 fusion protein disclosed herein or a pharmaceutical composition thereof that is administered with an additional therapeutic agent. Such additional agents include, but are not limited to, cytotoxic agents, chemotherapeutic agents, growth inhibitory agents, anti-inflammatory agents, anti-cancer agents, anti-neurodegenerative agents, and anti-infective agents. Agents that are used in such combination therapies may fall into one or more of the preceding categories. The administration of the IL-7/IL-21 fusion proteins and the additional therapeutic agent may be concurrent or consecutive. The administration of the IL-7/IL-21 fusion proteins and the additional therapeutic agent may be separately or as a mixture. Further, the methods of treatment contemplated herein can relate to a treatment in combination with one or more cancer therapies selected from the group of antibody therapy, chemotherapy, cytokine therapy, dendritic cell therapy, gene therapy, hormone therapy, laser light therapy, and radiation therapy.

Non-limiting examples of additional therapeutic agents also include radionuclides with high-energy ionizing radiation that are capable of causing multiple strand breaks in nuclear DNA, and therefore suitable for inducing cell death (e.g., of a cancer). Non-limiting examples of high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Non-limiting examples of additional therapeutic agents also include cytotoxic agents such as cytostatics (e.g., alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Non-limiting examples of additional therapeutic agents also include alkylating agents such as the anthracycline family of drugs (e.g., adriamycin, carminomycin, cyclosporin-A, chloroquine, methopterin, mithramycin, porfiromycin, streptonigrin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Non-limiting examples of DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Non-limiting examples of additional therapeutic agents also include transcription regulators such as actinomycin D, daunorubicin, doxorubicin, homoharringtonine, and idarubicin. Other non-limiting examples of cytostatic agents that are compatible with the embodiments disclosed herein include ansamycin benzoquinones, quinonoid derivatives (e.g., quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g., carmustine, lomustine, semustine).

Non-limiting examples of additional therapeutic agents also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g., paclitaxel, docetaxel, taxane), nocodazole, rhizoxin, dolastatins (e.g., Dolastatin-10, -11, or -15), colchicine and colchicinoids (e.g., ZD6126), combretastatins (e.g., Combretastatin A-4, AVE-6032), and vinca alkaloids (e.g., vinblastine, vincristine, vindesine, and vinorelbine (navelbine)); anti-angiogenesis compounds such as Angiostatin K1-3, DL-α-difluoromethyl-ornithine, endostatin, fumagillin, genistein, minocycline, staurosporine, and (±)-thalidomide.

Non-limiting examples of additional therapeutic agents also include hormones and hormone antagonists, such as corticosteroids (e.g., prednisone), progestins (e.g., hydroxyprogesterone or medroprogesterone), estrogens, (e.g., diethylstilbestrol), antiestrogens (e.g., tamoxifen), androgens (e.g., testosterone), aromatase inhibitors (e.g., aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Non-limiting examples of additional therapeutic agents also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Non-limiting examples of additional therapeutic agents also include gene regulators such as 5-aza-2′-deoxycytidine, 5-azacytidine, cholecalciferol (vitamin D3), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Non-limiting examples of additional therapeutic agents also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other additional therapeutic agents that are compatible with the teachings herein include auristatins (e.g., auristatin E and monomethylauristan E), calicheamicin, gramicidin D, maytansanoids (e.g., maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

In one embodiment, the IL-7/IL-21 fusion protein is administered in combination with an agent that is a checkpoint inhibitor. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD-1, B7-H3, B7-H4, BTLA, HVEM, GAL9, LAG3, TIM-3, VISTA, KIR, 2B4 (belongs to the CD2 family of molecules and is expressed on all NK, γδ, and memory CD8⁺ (αβ3) T cells), CD160 (also referred to as BY55), CGEN-15049, CHK1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-1-16 and B7-117. Checkpoint inhibitors include antibodies, or antigen binding fragments thereof, other binding proteins, biologic therapeutics or small molecules, that bind to and block or inhibit the activity of one or more of CTLA-4, PDL1, PDL2, PD-1, BTLA, HVEM, TIM-3, GAL9, LAG3, VISTA, KIR, 2B4, CD160 and CGEN-15049. Illustrative immune checkpoint inhibitors include Tremelimumab (CTLA-4 blocking antibody), anti-OX40, and Yervoy/ipilimumab (anti-CTLA-4 checkpoint inhibitor), as well as the PD-1 and PD-L1 inhibitors described herein. Checkpoint protein ligands include, but are not limited to PD-L1, PD-L2, B7-H13, B7-H4, CD28, CD86 and TIM-3.

In some embodiments, the IL-7/IL-21 fusion protein described herein are administered with a TIGIT, LAP, Podoplanin, Protein C receptor, ICOS, GITR, CD226 or a CD160 inhibiting agent.

### Kits

Provided herein are kits that comprise an IL-7/IL-21 fusion protein disclosed herein, a nucleic acid encoding an IL-7/IL-21 fusion protein disclosed herein, a oncolytic virus comprising a nucleic acid encoding an IL-7/IL-21 fusion protein disclosed herein, or an immune cell expressing on its surface an IL-7/IL-21 fusion protein disclosed herein. Provided herein are kits that comprise a pharmaceutical composition comprising an IL-7/IL-21 fusion protein disclosed herein, a nucleic acid encoding an IL-7/IL-21 fusion protein disclosed herein, a oncolytic virus comprising a nucleic acid encoding an IL-7/IL-21 fusion protein disclosed herein, or an immune cell expressing on its surface an IL-7/IL-21 fusion protein disclosed herein or a pharmaceutical composition thereof.

In some embodiments, the kit further includes one or more components such as instructions for use, devices and additional reagents, and components, such as tubes, containers and syringes for performing the methods disclosed herein.

In some embodiments, the kit includes instructions for use, a device for administering the IL-7/IL-21 fusion protein, the nucleic acid encoding a IL-7/IL-21 fusion protein, the oncolytic virus comprising a nucleic acid encoding a IL-7/IL-21 fusion, or the immune cell expressing on its surface an IL-7/IL-21 fusion or a pharmaceutical composition thereof to a subject, or a device for administering an additional agent or compound to a subject. For example, and not by way of limitation, the instructions can include a description of the IL-7/IL-21 fusion protein, the nucleic acid encoding the IL-7/IL-21 fusion protein, the oncolytic virus comprising a nucleic acid encoding a IL-7/IL-21 fusion, or the immune cell expressing on its surface an IL-7/IL-21 fusion or a pharmaceutical composition thereof and, optionally, other components included in the kit, and methods for administration, including methods for determining the proper state of the subject, the proper dosage amount and the proper administration method for administering the modified virus. Instructions can also include guidance for monitoring the subject over duration of the treatment time.

In some embodiments, the kit includes a device for administering the IL-7/IL-21 fusion protein, the nucleic acid encoding the IL-7/IL-21 fusion protein, the oncolytic virus comprising a nucleic acid encoding a IL-7/IL-21 fusion, or the immune cell expressing on its surface an IL-7/IL-21 fusion or a pharmaceutical composition thereof to a subject. Any of a variety of devices known in the art for administering medications and pharmaceutical compositions can be included in the kits provided herein. For example, and not by way of limitation, such devices include, a hypodermic needle, an intravenous needle, a catheter, a needle-less injection device, an inhaler and a liquid dispenser, such as an eyedropper. In certain embodiments, a modified virus to be delivered systemically, for example, by intravenous injection, can be included in a kit with a hypodermic needle and syringe.

In some embodiments, the kit includes one or more additional agents that can be administered in combination with the IL-7/IL-21 fusion protein disclosed herein, the nucleic acid encoding an IL-7/IL-21 fusion protein disclosed herein, the oncolytic virus comprising a nucleic acid encoding an IL-7/IL-21 fusion protein disclosed herein, or the immune cell expressing on its surface an IL-7/IL-21 fusion protein disclosed herein or the pharmaceutical composition thereof. For example, but not by way of limitation, a kit can include a cytokine, e.g., IL-2, and/or an anti-PD-1 and/or an anti-PD-L1 antibody.

It is to be understood that this disclosure is not limited to the particular molecules, compositions, methodologies, or protocols described, as these may vary. Any methods and materials similar or equivalent to those described herein can be used in the practice or testing of embodiments disclosed herein. It is further to be understood that this disclosure includes all possible combinations of such particular features. For example, where a particular feature is disclosed in the context of a particular aspect or embodiment of the disclosure, or a particular claim, that feature can also be used, to the extent possible, in combination with and/or in the context of other particular aspects and embodiments disclosed herein.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present disclosure, the following examples of specific embodiments are given. The following examples should not be read to limit or define the entire scope of the disclosure.

### EXAMPLES

### Example 1: Material and Methods for Examples 1-5

### Mice and Cell Lines

Female C57BL/6 (B6 in short) mice were purchased from The Jackson Laboratory (Bar Harbor, ME) and housed in specific pathogen-free conditions in Allegheny Health Network Research Institute Preclinical Facility. All animal studies were approved Allegheny Health Network Research Institute Institutional Animal Care and Use Committee. Mouse colon cancer MC38-luc, ovarian cancer ID8a-luc, and mesothelioma AB12-luc cells were generated by the infection of parental tumor cells with firefly luciferase-carrying lentivirus and antibiotic blasticidin selection. Normal African green monkey kidney fibroblast CVI, Human embryonic kidney 293 (HEK293) cells, and mouse melanoma B16 cells were obtained from American Type Culture Collection. HEK293 cells were grown in Dulbecco's Modified Eagle's medium (DMEM) supplemented with 20% calf bovine serum (CBS), 2 mM L-glutamine, and 1× penicillin/streptomycin in a 37° C., 5% CO₂ incubator. Other cell lines were grown in DMEM supplemented with 10% fetal bovine serum (FBS), 2 mM L-glutamine, and 1×penicillin/streptomycin in a 37° C., 5% CO₂ incubator.

### Virus Generation

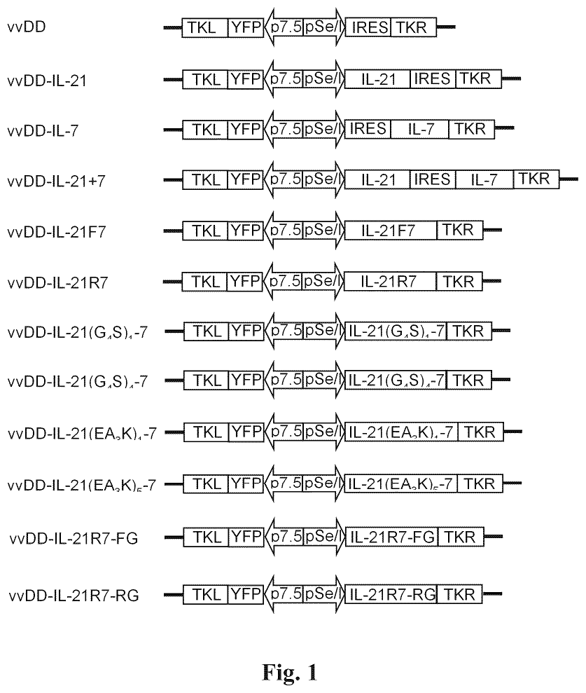

VSC20, a vgf gene-deleted Western Reserve strain vaccinia virus, was used as the parental virus for homologous recombination. Murine IL-21 and/or IL-7 cDNAs were PCR-amplified from plasmids pMD-18-IL-21 and pMD-18-IL-7 (Cat: MG50137-M and MG50217-NM, respectively; SinoBiological US, Wayne, PA) and inserted into pCMS1-IRES, pCMS1-IRES-FGI or pCMS1-IRES-RGPI to generate pCMS1-shuttle plasmids for the expression of the proteins shown in FIG. 1 and Table 1. The primers for plasmid cloning based on PCR are listed in Table 2. The glycosylphosphatidylinositol (GPI) anchor sequences were derived from human CD16b Val196-I1e233 and fused to the C-terminus of IL-21R7 to generate cell membrane-bound proteins (see Table 3 for sequence). To make new viruses, CV-1 cells were infected with VSC20 at a MOI of 0.1 and then transfected with the shuttle plasmids, resulting in virus mixture of parental virus and recombinant virus, respectively. Selection of these new recombinant viruses was based on expression of yellow fluorescent protein in CVI cells 24 hours post-infection of relative virus mixture. vvDD-YFP, or vvDD for short, a double viral gene-inactivated (tk- and vgf-) vaccinia virus carrying yfp cDNA at the tk locus, was the control virus for this work. All genomes contained an inactivated thymidine kinase (TK) gene. TK is involved in the synthesis of deoxyribonucleotides. The viral TK gene is not required for dividing cells, while is essential for infection of resting cells. Disruption of the vaccinia virus TK gene leads to significant attenuation in normal tissues, while dividing cells and tumor tissues are able to complement this gene deletion and support viral replication.

TABLE 1
Viral vectors for the expression of IL-7, IL-21,
or IL-7/IL-21 fusion proteins, respectively.

| Viral vector | Protein to be expressed |
|---|---|
| vvDD | — |
| vvDD-IL-21 | IL-21 |
| vvDD-IL-7 | IL-7 |
| vvDD-IL-21 + 7 | IL-21 and IL-7 (expressed separately) |
| Viral vector | Fusion protein to be expressed |
| vvDD-IL-21F7 | IL-21 - (G₄S)₃ (flexible linker) - IL-7 |
| vvDD-IL-21R7 | IL-21 - A(EA₃K)₄AAA (rigid linker) IL-7 |
| vvDD-IL-21-(G₄S)₁-7 | IL-21 - (G₄S)₁ (flexible linker) - IL-7 |
| vvDD-IL-21-(G₄S)₄-7 | IL-21 - (G₄S)₄ (flexible linker) - IL-7 |
| vvDD-IL-21(EA₃K)₁-7 | IL-21 - (EA₃K)₁ (rigid linker) - IL-7 |
| vvDD-IL-21(EA₃K)₅-7 | IL-21 - (EA₃K)₅ (rigid linker) - IL-7 |
| vvDD-IL-21R7-FG | IL-21 - A(EA₃K)₄AAA (rigid linker) - IL-7 - |
| | (G₄S)₃ (flexible linker) - GPI anchor |
| vvDD-IL-21R7-RG | IL-21 - A(EA₃K)₄AAA (rigid linker) - IL-7 - |
| | A(EA₃K)₄AAA (rigid linker) - GPI anchor |

### The Expression of Gene of Interest In Vitro

MC38-luc (3×10⁵), AB12-luc (3×10⁵), or B16 (2×10⁵) cells were seeded in 24-well plates overnight and mock-infected or infected with vvDD, vvDD-IL-21, vvDD-IL-7, and vvDD-IL-21+7, vvDD-IL-21F7 or vvDD-IL-21R7 at an MOI of 1 in 0.15 mL 2% FBS-containing-DMEM for 2 h, respectively. 350 μL 10% FBS-containing-DMEM was added to cells and the cells were cultured until harvest at 24 h post-viral infection. The cell pellets were applied to extract RNA to measure the viral house-keeping gene A34R, transgene IL-21 or IL-7 using RT-qPCR, respectively. One microgram of RNA was used for cDNA synthesis, and 25 to 50 ng of subsequent cDNA was used to conduct mRNA expression TaqMan analysis on the Quantagene q225 qPCR System (Kubo Technology, Beijing, China). All primers for the analysis were purchased from Thermo Fisher Scientific (Waltham, Massachusetts). Gene expression was normalized to the housekeeping gene HPRT1 and expressed as fold increase (2−ΔCT), where ΔCT=CT(target gene)−CT (HPRT1).

### Rodent Tumor Models

B6 mice were intraperitoneally (i p.) inoculated with 5×10⁵ MC38-luc cancer cells or 3.5×10⁵ ID8A-luc, respectively, and divided into required groups at the indicated day post-tumor cell inoculation according to tumor size based on live animal IVIS imaging, performed using a Xenogen IVIS 200 optical in vivo imaging system (Caliper Life Sciences, Hopkinton, MA). Grouped mice were i.p. injected with indicated viruses, or PBS.

MC38-luc-tumor-bearing B6 mice treated with vvDD-IL-21, vvDD-IL-21F7 or vvDD-IL-21R7, respectively, which had survived for more than 150 days, were subcutaneously (s.c.) challenged with 1×10⁶ parental MC38 cancer cells in the left flanks and 3×105 B16 cells in the right flanks, respectively. Naïve B6 rice also received the same dose tumor challenge as a control. Subcutaneous tumor size was measured using an electric caliper in two perpendicular diameters.

### Statistics

Statistical analyses were performed using unpaired Student's t test (GraphPad Prism version 9). Data are means SD. Animal survival is presented using Kaplan-Meier survival curves and was statistically analyzed using a log-rank test (GraphPad Prism version 9). Tumor growth cures were statistically analyzed using two-way ANOVA (GrapPad Prism version 9). Values of P<0.05 were considered statistically significant, and all P values were two-sided. In the figures, standard symbols are used: *P<0.05; **P<0.01; ***P<0.001; and ****P<0.0001.

### Example 2: Viral Vector Replication and Cytokine Expression in Tumor Cells

To demonstrate that oncolytic vaccinia viral vectors are useful for the delivery of genes for the expression of cytokines in tumor cells, the cells were (i) mock-infected or infected with (ii) vvDD or (iii) vvDD expressing IL-21 (vvDD-IL-21), (iv) vvDD expressing IL-7 (vvDD-IL-7), (v) vvDD expressing IL-21 and IL-7 separately (vvDD-IL-21+7), (vi) vvDD expressing IL-21 fused to IL-7 with a flexible linker (G₄S)₃ (IL-21F7), and (vii) vvDD expressing IL-21 fused to IL-7 with a rigid linker A(EA₃K)₄AAA (IL-21R7), respectively, at a multiplicity of infection (MOI) of 1. The following tumor cell lines were used: MC38-luc (3×10⁵ cells), a mouse colon cancer cell line expressing luciferase, B16 (2×10⁵ cells), a mouse melanoma cell line, and AB12-luc (3×10⁵ cells), a mouse mesothelioma cell line expressing luciferase.

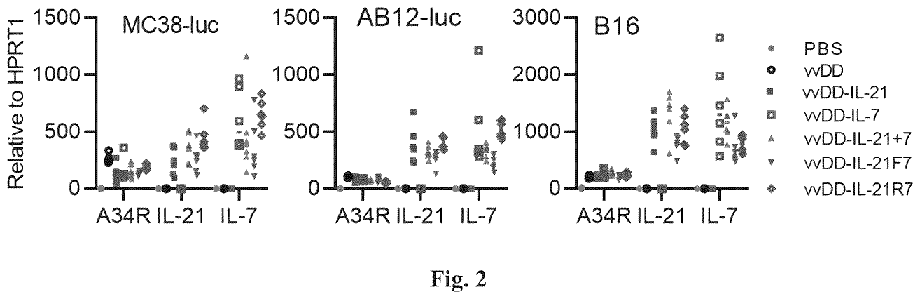

Expression of control gene A34 (a viral housekeeping gene encoding a vaccinia outer envelope protein), IL-7, and IL-21 was determined by RT-qPCR (FIG. 2). Viral housekeeping gene (A34R) mRNA levels in cell pellets were similar in the cells receiving the treatment of these viruses, while IL-21 mRNA levels in cell pellets were significantly higher in the cells receiving IL-21 incorporated viruses than non-IL-21 incorporated viruses. The same was true for IL-7 expression levels.

All vectors expressed their respective transgenes, indicating that the designed viral vectors are useful for the delivery of genes for the expression of cytokines IL-7 and IL-21, as well as a fusion protein comprising IL-21 and IL-7, to different tumor cells.

### Example 3: Oncolytic Vaccinia Viral Vectors Expressing Fusion Proteins Comprising IL-7 and IL-21 Elicit Potent Therapeutic Effects in Murine Cancer Models

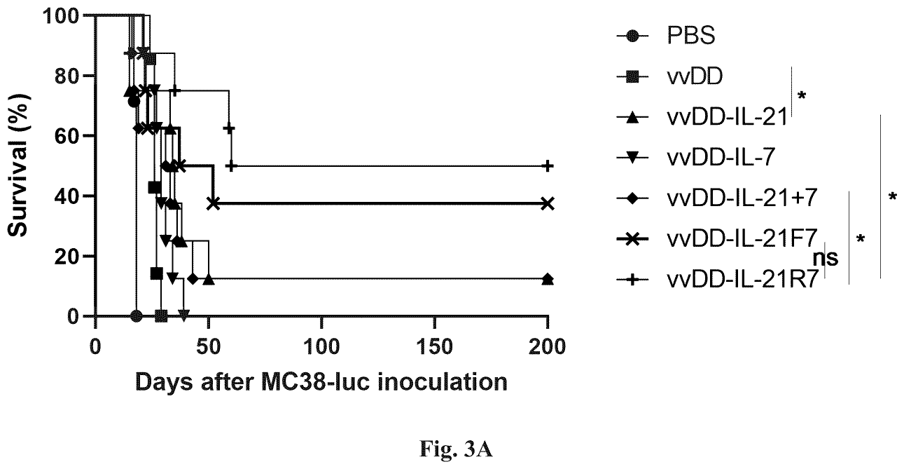

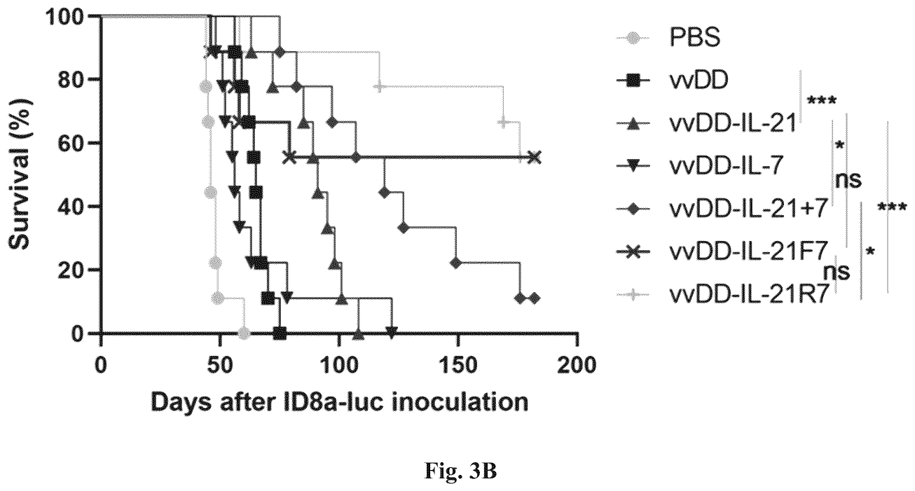

To assess the anti-tumor activity of fusion proteins comprising IL-7 and IL-21, C57BL/6 mice were inoculated i.p. with 5×10⁵ MC38-luc cells (FIG. 3A) or 3.5×10⁶ ovarian cancer ID8A-luc cells (FIG. 3B), respectively. The mice were treated with (i) PBS, (ii) vvDD, or (iii) vvDD expressing IL-21 (vvDD-IL-21), (iv) vvDD expressing IL-7 (vvDD-IL-7), (v) vvDD expressing IL-21 and IL-7 separately (vvDD-IL-21-+7), (vi) vvDD expressing IL-21 fused to IL-7 with a flexible linker (G₄S)₃ (IL-21F7), and (vii) vvDD expressing IL-21 fused to IL-7 with a rigid linker A(EA₃K)₄AAA (IL-21R7), respectively, at 2×10⁸ PFU/mouse nine days after tumor inoculation. Mouse survival was determined.

Fusion proteins IL-21F7 or IL-21R7 elicited more potent antitumor effects in the colon cancer model, resulting in increased survival, as compared to IL-21, IL-7, or a combination of IL-21 and IL-7, in which the two cytokines were delivered as two separate proteins (FIG. 3A). The median survival days were 34.5, 32, 44.5 or 130 after the treatment of with vvDD expressing IL-21 (vvDD-IL-21), vvDD expressing IL-21 and IL-7 separately (vvDD-IL-21+7), IL-21 fused to IL-7 with a flexible linker (G₄S)₃ (IL-21F7), or IL-21 fused to IL-7 with a rigid linker A(EA₃K)₄AAA (IL-21R7), respectively.

Fusion proteins IL-21 F7 or IL-21R7 also elicited potent antitumor effects compared in the ovarian cancer model (FIG. 3B). The median survival days were 91, 119, >182 or >182 after the treatment with vvDD expressing IL-21 (vvDD-IL-21), vvDD expressing IL-21 and IL-7 separately (vvDD-IL-21-7), vvDD expressing IL-21 fused to IL-7 with a flexible linker (G₄S)₃ (IL-21F7), or vvDD expressing IL-21 fused to IL-7 with a rigid linker A(EA₃K)₄AA A (IL-21 R7), respectively.

### Example 4: Oncolytic Vaccinia Viral Vectors Expressing Fusion Proteins Comprising IL-7 and IL-21 Elicit Potent Therapeutic Effects in at the Memory Phase

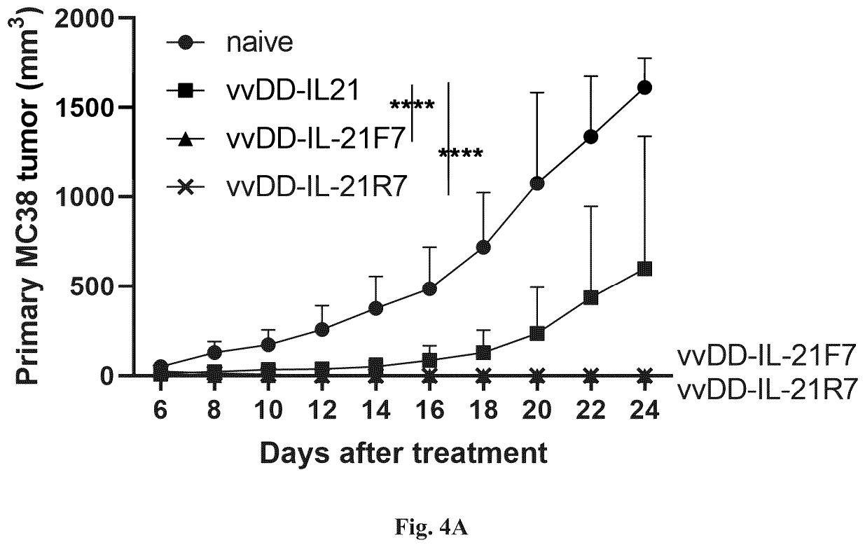

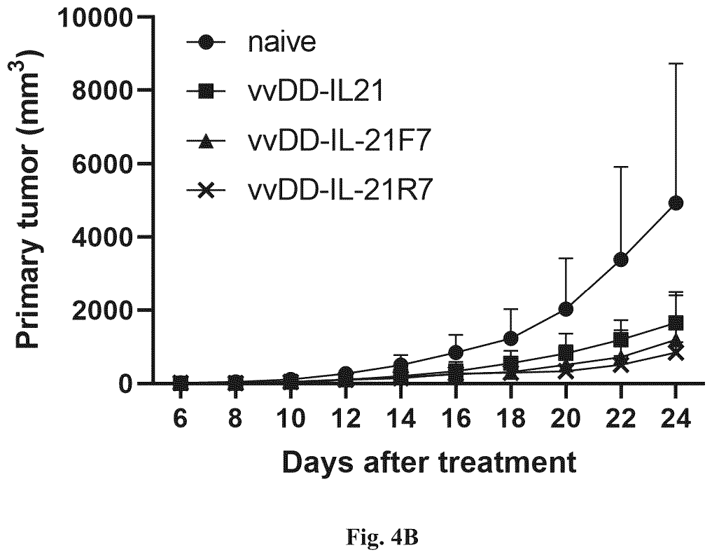

To demonstrate that oncolytic vaccinia viral vectors expressing fusion protein IL-21F7 or IL-21R7, respectively, elicit potent tumor-specific antitumor effects at the memory phase, MC38-luc-intraperitoneal-bearing C57BL/6 mice which were treated with (i) PBS, (ii) vvDD expressing IL-21 (vvDD-IL-21), (iii) vvDD expressing IL-21 fused to IL-7 with a flexible linker (G₄S)₃ (IL-21F7), and (iv) vvDD expressing IL-L21 fused to IL-7 with a rigid linker A(EA₃K)₄AAA (IL-21R7), respectively. Mice that survived more than 150 days were s.c. injected with 1×10⁶ MC38 cells in the left flanks (FIG. 4A) and 3×10⁵ B16 cells in the right flanks (FIG. 4B), respectively. Tumor growth cures were determined. The mice cured with vvDD-IL-21F7 or vvDD-IL-21R7 rejected MC38 re-challenge (FIG. 4A). For both types of tumors, fusion proteins IL-21F7 or IL-21R7 elicited more potent antitumor effects, resulting in reduced tumor growth, as compared to IL-21 alone.

### Example 5: Effects of Anti-CD8, Anti-CD4, Anti-IFN-γ and Anti-CD117 Antibodies on Antitumor Effects Elicited by vvDD-IL-21R7

To determine whether the antitumor effects elicited by vvDD-IL-21R7 are dependent on CD8⁺ cells, CD4⁺ T cells, mast cells (CD117) and IFN-γ, C57BL/6 mice were inoculated i.p. with 5×10⁵ MC38-luc cells and treated with PBS or vvDD expressing IL-21 fused to I1-7 with a rigid linker A(EA₃K)₄AAA (IL-21 R7) at 2×10⁸ PFU/mouse nine days after tumor inoculation. Anti-CD8, anti-CD4, anti-IFN-γ, and anti-CD117 antibodies were administered to the mice which had received vvDD-IL-21R7 as follows: (1) α-CD8 antibodies (150 μg/injection) were injected at Day 5, 7, 9 and 11 after viral treatment, (2) α-CD4 antibodies (150 μg/injection) were injected at Day 5, 7, 9 and 11 after viral treatment, (3) α-IFN-γ antibodies (200 μg/injection) were injected at Day 5, 7, 9 and 11 after viral treatment, or (4) α-CD117 antibodies (200 μg/injection) were injected at Day 9, 11, 13 and 14 after tumor cell injection, respectively. Mouse survival was determined.

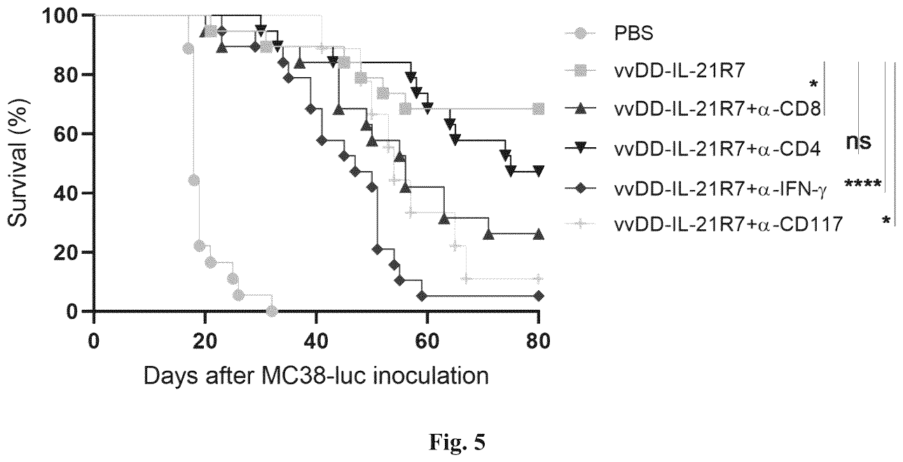

The antitumor effects elicited by vvDD-IL-21R7 were dependent on CD8⁺ T cells, mast cells (CD117⁺) and IFN-γ (FIG. 5).

### Example 6: Linker Length

To show that the fusion proteins described here may comprise a variety of different linkers connecting IL-21 and 11-7, C57BL/6 mice were inoculated i.p. with 5×10⁵ MC38-luc cells. Nine days after tumor inoculation, the mice were treated with (i) PBS, (ii) vvDD expressing IL-21 and IL-7 separately (vvDD-IL-21-7), (iii) vvDD expressing IL-21 fused to IL-7 with flexible linker (G₄S)₃ (IL-21F7), (iv) vvDD expressing IL-21 fused to IL-7 with rigid linker A(EA₃K)₄AAA (IL-21R7), (v) vvDD expressing IL-21 fused to IL-7 with flexible linker (G₄S)₁ (IL-21-(G₄S)₁-7), (vi) vvDD expressing IL-21 fused to IL-7 with flexible linker (G₄S)₄ (IL-21-(G₄)₄-7), (vii) vvDD expressing IL-21 fused to IL-7 with rigid linker (EA₃K)₁ (IL-21(EA₃K)₅-7), and (viii) vvDD expressing IL-21 fused to IL-7 with rigid linker (EA₃K)₅ (IL-21(EA₃K₅-7) (at a dose of 2×10⁸ PFU per mouse).

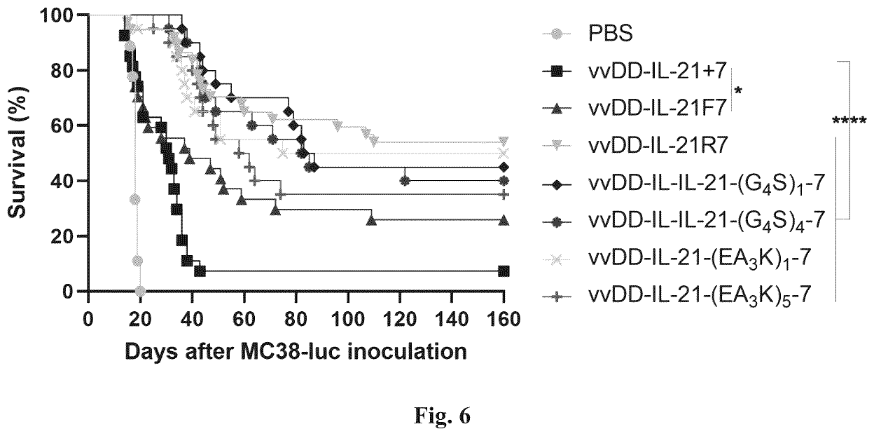

Survival results demonstrated that all fusion cytokine-armed vvDD elicited potent antitumor effects compared with PBS or vvDD expression IL-21 and IL-7 separately in the colon cancer model (FIG. 6).

### Example 7: Generation of Membrane-Associated Fusion Cytokines

To test the ability of membrane-associated fusion cytokines to elicit anti-tumor effects, PBS or constructs vvDD-IL-21R7, vvDD-IL-21R7-FG, or vDD-IL-21R7-RG, respectively, were injected at the dose of 2×10⁸ PFU per mouse i.p., into B6 mice bearing nine-day old peritoneal murine colon cancer MC38-luc. Construct IL-21R7-FG comprises IL-21, a rigid linker (A(EA₃K)₄AAA), IL-7, a flexible linker (GGGGS)₃, and a GPI anchor. Construct IL-21R7-RG comprises IL-21, a first rigid linker A(EA₃K)₄AAA, IL-7, a second rigid linker A(EA₃K)₄AAA, and a GPI anchor.

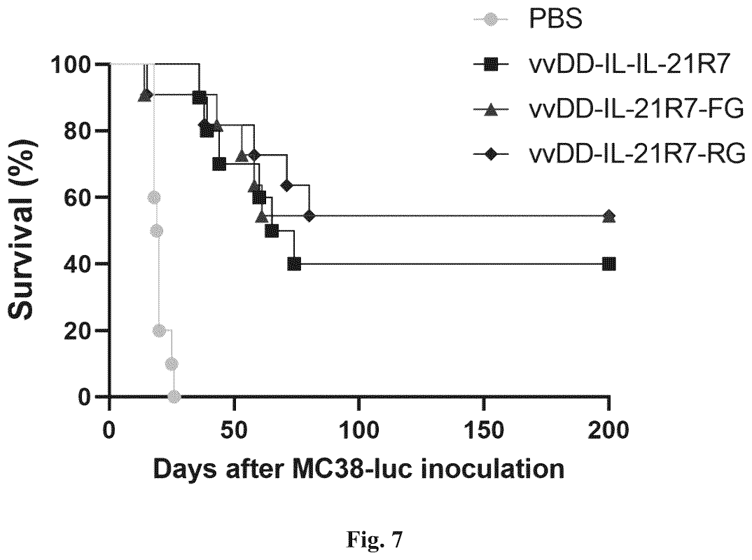

Survival results demonstrated that the vvDD expressing the soluble form of IL-21 fused to IL-7 with rigid linker A(EA₃K)₄AAA (IL-21 R7) elicited similar antitumor effects as compared with the membrane-associated fusion cytokines in a colon cancer model (FIG. 7).

TABLE 2
Primers.

| Primer | | SEQ | | | |
|---|---|---|---|---|---|
| No. | Primer (5′ to 3′) | ID NO | PCR Template | Cloning Plasmid | Resulting Plasmid |
| P1 | GGCGGTCGACATGGAGAGGACCCTTG | 1 | pMD-18-IL-21 | pCMS1-IRES | pCMS1-IL-21-IRES |
| | TCTGTC | | | | |
| P2 | CGCGGGCGCGCCCTAGGAGAGATGCT | 2 | | | |
| | GATGAATC | | | | |
| P1 | GGCGGTCGACATGGAGAGGACCCTTG | 1 | pMD-18-IL-21 | pCMS1-IRES-FGPI | pCMS1-IL-21-FGPI |
| | TCTGTC | | | | |
| P3 | CCGCGGCCGGCCCGGAGAGATGCTGA | 3 | | | |
| | TGAATC | | | | |
| P4 | GGCGGTCGACATGTTCCATGTTTCTT | 4 | pMD-18-IL-7 | pCMS1-IRES | pCMS1-IRES-IL-7 |
| | TTAGATATATC | | | | |
| P5 | CGCGGTTAACTTATATACTGCCCTTC | 5 | | | |
| | AAAATTTTATTC | | | | |
| P6 | GGCGCCTGCAGGATGTTCCATGTTTC | 6 | pMD-18-IL-7 | pCMS1-IL-21-IRES | pCMS1-IL-21-IRES-IL-7 |
| | TTTTAGATATATC | | | | |
| P5 | CGCGGTTAACTTATATACTGCCCTTC | 5 | | | |
| | AAAATTTTATTC | | | | |
| P7 | GGCCGGCCGGCCGGTGGCGGTGGGAG | 7 | pMD-18-IL-7 | pCMS1-IL-21-FGPI | pCMS1-IL-21F7 |
| | CGGTGGTGGGGGTTCCGGAGGCGGAG | | | | |
| | GGTCG | | | | |
| P8 | GGTTCCGGAGGCGGAGGGTCGGAGTG | 8 | | | |
| | CCACATTAAAGACAAAG | | | | |
| P5 | CGCGGTTAACTTATATACTGCCCTTC | 5 | | | |
| | AAAATTTTATTC | | | | |
| P9 | GGCCGGCCGGCCGCTGAAGCTGCCGC | 9 | pMD-18-IL-7 | pCMS1-IL-21-FGPI | pCMS1-IL-21R7 |
| | AAAAGAGGCCGCTGCGAAGGAGGCCG | | | | |
| | CGGCTAAG | | | | |
| P10 | GAGGCCGCGGCTAAGGAGGCGGCAGC | 10 | | | |
| | TAAAGCTGCAGCCGAGTGCCACATTA | | | | |
| | AAGACAAA | | | | |
| P5 | CGCGGTTAACTTATATACTGCCCTTC | 5 | | | |
| | AAAATTTTATTC | | | | |
| P11 | GATCGGCCGGCCGGTGGCGGTGGGAG | 64 | pCMS1-IL-21F7 | pCMS1-IL-21-FGPI | pCMS1-IL-21(G₄S)₁-7 |
| | CGAGTGCCACATTAAAGACAAAGAAG | | | | |
| | G | | | | |
| P5 | CGCGGTTAACTTATATACTGCCCTTC | 5 | | | |
| | AAAATTTTATTC | | | | |
| P12 | GATCGGCCGGCCGGTGGTGGGGGTTC | 65 | pCMS1-IL-21F7 | pCMS1-IL-21-FGPI | pCMS1-IL-21(G₄S)₄-7 |
| | GGGTGGGGGGGGAGCGGTGGTGGGG | | | | |
| | GTTCCGG | | | | |
| P5 | CGCGGTTAACTTATATACTGCCCTTC | 5 | | | |
| | AAAATTTTATTC | | | | |
| P13 | GATCGGCCGGCCGCTGAGGCGGCAGC | 66 | pCMS1-IL-21R7 | pCMS1-IL-21-FGPI | pCMS1-IL-21(EA₃K)₅-7 |
| | TAAAGCTGCAGCCGAGTGCCACATTA | | | | |
| | AAGAC | | | | |
| P5 | CGCGGTTAACTTATATACTGCCCTTC | 5 | | | |
| | AAAATTTTATTC | | | | |
| P14 | GATCGGCCGGCCGCTGAAGCTGCCGC | 67 | pCMS1-IL-21R7 | pCMS1-IL-21-FGPI | pCMS1-IL-21(EA₃K)₅-7 |
| | TAAGGAAGCTGCCGCAAAAGAGGCCG | | | | |
| | CTGCGAAG | | | | |
| P5 | CGCGGTTAACTTATATACTGCCCTTC | 5 | | | |
| | AAAATTTTATTC | | | | |
| P15 | TAAGCTCGAAGTCGACATGGAGAGGA | 68 | pCMS1-IL-21R7 | pCMS1-IRES-FGPI | pCMS1-IL-21R7-FG |
| | CCCTTGTCT | | | | |
| P16 | TCCCACCGCCACCGGCCGGCCCTATA | 69 | | | |
| | CTGCCCTTCAAAATTTTATT | | | | |
| P15 | TAAGCTCGAAGTCGACATGGAGAGGA | 68 | pCMS1-IL-21R7 | pCMS1-IRES-RGPI | pCMS1-IL-21R7-RG |
| | CCCTTGTCT | | | | |
| P17 | CGGCAGCTTCAGCGGCCGGCCCTATA | 70 | | | |
| | CTGCCCTTCAAAATTTTATT | | | | |

Restriction sites are underlined.

TABLE 3
Amino acid sequences for IL-7, IL-21, and IL-7/IL-21 fusion proteins.

| SEQ | | |
|---|---|---|
| ID | | |
| NO | Description | Sequence |
| 11 | Murine IL-21 with | MERTLVCLVVIFLGTVAHKSSPQGPDRLLIRLRHLIDIVEQLKIYENDLDPELLSAPQDVKGHCEHAAFACFQ |
| | signal sequence | KAKLKPSNPGNNKTFIIDLVAQLRRRLPARRGGKKQKHIAKCPSCDSYEKRTPKEFLERLKWLLQKMIHQHLS |
| 12 | Murine IL-21 w/o | QGPDRLLIRLRHLIDIVEQLKIYENDLDPELLSAPQDVKGHCEHAAFACFQKAKLKPSNPGNNKTFIIDLVAQ |
| | signal sequence | LRRRLPARRGGKKQKHIAKCPSCDSYEKRTPKEFLERLKWLLQKMIHQHLS |
| 13 | Murine IL-7 with | MFHVSFRYIFGIPPLILVLLPVTSSECHIKDKEGKAYESVLMISIDELDKMTGTDSNCPNNEPNFFRKHVCDD |
| | signal sequence | TKEAAFLNRAARKLKQFLKMNISEEFNVHLLTVSQGTQTLVNCTSKEEKNVKEQKKNDACFLKRLLREIKTCW |
| | | NKILKGSI |
| 14 | Murine IL-7 w/o signal | ECHIKDKEGKAYESVLMISIDELDKMTGTDSNCPNNEPNFFRKHVCDDTKEAAFLNRAARKLKQFLKMNISEE |
| | sequence | FNVHLLTVSQGTQTLVNCTSKEEKNVKEQKKNDACFLKRLLREIKTQWNKILKGSI |
| 15 | Murine IL-21F7 | MERTLVCLVVIFLGTVAHKSSPQGPDRLLIRLRHLIDIVEQLKIYENDLDPELLSAPQDVKGHCEHAAFACFQ |
| | | KAKLKPSNPGNNKTFIIDLVAQLRRRLPARRGGKKQKHIAKCPSCDSYEKRTPKEFLERLKWLLQKMIHQHLS |
| | | GPAGGGGSGGGGSGGGGSECHIKDKEGKAYESVLMISIDELDKMTGTDSNCPNNEPNFFRKHVCDDTKEAAFL |
| | | NRAARKLKQFLKMNISEEENVHLLTVSQGTQTLVNCTSKEEKNVKEQKKNDACFLKRLLREIKTCWNKILKGS |
| | | I |
| 16 | Murine IL-21F7 w/o | MERTLVCLVVIFLGTVAHKSSPQGPDRLLIRLRHLIDIVEQLKIYENDLDPELLSAPQDVKGHCEHAAFACFQ |
| | “GPA” | KAKLKPSNPGNNKTFIIDLVAQLRRRLPARRGGKKQKHIAKCPSCDSYEKRTPKEFLERLKWLLQKMIHQHLS |
| | | GGGSGGGGSGGGGSECHIKDKEGKAYESVLMISIDELDKMTGTDSNCPNNEPNFFRKHVCDDTKEAAFLNRAA |
| | | RKLKQFLKMNISEEFNVHLLTVSQGTQTLVNCTSKEEKNVKEQKKNDACFLKRLLREIKTCWNKILKGSI |
| 17 | Murine IL-21R7 | MERTLVCLVVIFLGTVAHKSSPQGPDRLLIRLRHLIDIVEQLKIYENDLDPELLSAPQDVKGHCEHAAFACFQ |
| | | KAKLKPSNPGNNKTFIIDLVAQLRRRLPARRGGKKQKHIAKCPSCDSYEKRTPKEFLERLKWLLQKMIHQHLS |
| | | GPAAEAAAKEAAAKEAAAKEAAAKAAAECHIKDKEGKAYESVLMISIDELDKMTGTDSNCPNNEPNFFRKHVC |
| | | DDTKEAAFLNRAARKLKQFLKMNISEEFNVHLLTVSQGTQTLVNCTSKEEKNVKEQKKNDACFLKRLLREIKT |
| | | CWNKILKGSI |
| 18 | Murine IL-21R7 w/o | MERTLVCLVVIFLGTVAHKSSPQGPDRLLIRLRHLIDIVEQLKIYENDLDPELLSAPQDVKGHCEHAAFACFQ |
| | “GPA” | KAKLKPSNPGNNKTFIIDLVAQLRRRLPARRGGKKQKHIAKCPSCDSYEKRTPKEFLERLKWLLQKMIHQHLS |
| | | AEAAAKEAAAKEAAAKEAAAKAAAECHIKDKEGKAYESVLMISIDELDKMTGTDSNCPNNEPNFFRKHVCDDT |
| | | KEAAFLNRAARKLKQFLKMNISEEFNVHLLTVSQGTQTLVNCTSKEEKNVKEQKKNDACFLKRLLREIKTCWN |
| | | KILKGSI |
| 19 | Human IL-21 with | MRSSPGNMERIVICLMVIFLGTLVHKSSSQGQDRHMIRMRQLIDIVDQLKNYVNDLVPEFLPAPEDVETNCEW |
| | signal sequence | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDS |
| 20 | Human IL-21 w/o | QGQDRHMIRMRQLIDIVDQLKNYVNDLVPEFLPAPEDVETNCEWSAFSCFQKAQLKSANTGNNERIINVSIKK |
| | signal sequence | LKRKPPSTNAGRRQKHRLTCPSCDSYEKKPPKEFLERFKSLLQKMIHQHLSSRTHGSEDS |
| 21 | Human IL-7 with | MFHVSFRYIFGLPPLILVLLPVASSDCDIEGKDGKQYESVLMVSIDQLLDSMKEIGSNCINNEFNFFKRHICD |
| | signal sequence | ANKEGMFLFRAARKLRQFLKMNSTGDFDLHLLKVSEGTTILLNCTGQVKGRKPAALGEAQPTKSLEENKSLKE |
| | | QKKLNDLCFLKRLLQEIKTCWNKILMGTKEH |
| 22 | Human IL-7 w/o signal | DCDIEGKDGKQYESVLMVSIDQLLDSMKEIGSNCLNNEFNFFKRHICDANKEGMFLFRAARKLRQFLKMNSTG |
| | sequence | DFDLHLLKVSEGTTILLNCTGQVKGRKPAALGEAQPTKSLEENKSLKEQKKLNDLCFLKRLLQEIKTCWNKIL |
| | | MGTKEH |
| 23 | Human IL-21F7 | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAGGGGSGGGGSGGGGSDCDIEGKDGKQYESVLMVSIDQLLDSMKEIGSNCLNNEF |
| | | NFFKRHICDANKEGMFLFRAARKLRQFLKMNSTGDFDLHLLKVSEGTTILLNCTGQVKGRKPAALGEAQPTKS |
| | | LEENKSLKEQKKLNDLCFLKRLLQEIKTCWNKILMGTKEH |
| 24 | Human IL-21F7 w/o | MRSSPGNMERIVICLMVIFLGTLVHKSSSQGQDRHMIRMRQLIDIVDQLKNYVNDLVPEFLPAPEDVETNCEW |
| | “GPA” | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGGGGSGGGGSGGGGSDCDIEGKDGKQYESVLMVSIDQLLDSMKEIGSNCLNNEFNFF |
| | | KRHICDANKEGMFLFRAARKLRQFLKMNSTGDFDLHLLKVSEGTTILLNCTGQVKGRKPAALGEAQPTKSLEE |
| | | NKSLKEQKKLNDLCFLKRLLQEIKTCWNKILMGTKEH |
| 25 | Human IL-21R7 | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAAEAAAKEAAAKEAAAKEAAAKAAADCDIEGKDGKQYESVLMVSIDQLLDSMKEI |
| | | GSNCLNNEFNFFKRHICDANKEGMFLFRAARKLRQFLKMNSTGDFDLHLLKVSEGTTILLNCTGQVKGRKPAA |
| | | LGEAQPTKSLEENKSLKEQKKLNDLCFLKRLLQEIKTCWNKILMGTKEH |
| 26 | Human IL-21R7 w/o | RSSPGNMERIVICLMVIFLGTLVHKSSSQGQDRHMIRMRQLIDIVDQLKNYVNDLVPEFLPAPEDVETNCEWS |
| | “GPA” | AFSCFQKAQLKSANTGNNERIINVSIKKLKRKPPSTNAGRRQKHRLTCPSCDSYEKKPPKEFLERFKSLLQKM |
| | | IHQHLSSRTHGSEDSAEAAAKEAAAKEAAAKEAAAKAAADCDIEGKDGKQYESVLMVSIDQLLDSMKEIGSNC |
| | | LNNEFNFFKRHICDANKEGMFLFRAARKLRQFLKMNSTGDFDLHLLKVSEGTTILLNCTGQVKGRKPAALGEA |
| | | QPTKSLEENKSLKEQKKLNDLCFLKRLLQEIKTCWNKILMGTKEH |
| 27 | Example of a flexible | GGGGS |
| | linker | |
| 28 | Example of a flexible | GGSGGGS |
| | linker | |
| 29 | Example of a flexible | GGGGSGGGGS |
| | linker | |
| 30 | Example of a flexible | GGGGSGGGGSGGGGS |
| | linker | |
| 36 | Example of a flexible | GGGGSGGGGSGGGGSGGGGS |
| | linker | |
| 31 | Example of a rigid | AEAAAKEAAAKEAAAKEAAAKAAA |
| | linker | |
| 37 | Example of a rigid | AEAAAKAAA |
| | linker | |
| 38 | Example of a rigid | AEAAAKEAAAKEAAAKEAAAKEAAAKAAA |
| | linker | |
| 32 | Murine IL-21 signal | MERTLVCLVVIFLGTVAHKSSP |
| | sequence | |
| 33 | Murine IL-7 signal | MFHVSFRYIFGIPPLILVLLPVTSS |
| | sequence | |
| 34 | Human IL-21 signal | MRSSPGNMERIVICLMVIFLGTLVHKSSS |
| | sequence | |
| 35 | Human IL-7 signal | MFHVSFRYIFGLPPLILVLLPVASS |
| | sequence | |
| 39 | Murine IL-21(G₄S)₁-7 | MERTLVCLVVIFLGTVAHKSSPQGPDRLLIRLRHLIDIVEQLKIYENDLDPELLSAPQDVKGHCEHAAFACFQ |
| | | KAKLKPSNPGNNKTFIIDLVAQLRRRLPARRGGKKQKHIAKCPSCDSYEKRTPKEFLERLKWLLQKMIHQHLS |
| | | GPAGGGGSECHIKDKEGKAYESVLMISIDELDKMTGTDSNCPNNEPNFFRKHVCDDTKEAAFLNRAARKLKQF |
| | | LKMNISEEFNVHLLTVSQGTQTLVNCTSKEEKNVKEQKKNDACFLKRLLREIKTCWNKILKGSI |
| 40 | Murine IL-21(G₄S)₁-7 | MERTLVCLVVIFLGTVAHKSSPQGPDRLLIRLRHLIDIVEQLKIYENDLDPELLSAPQDVKGHCEHAAFACFQ |
| | w/o “GPA” | KAKLKPSNPGNNKTFIIDLVAQLRRRLPARRGGKKQKHIAKCPSCDSYEKRTPKEFLERLKWLLQKMIHQHLS |
| | | GGGGSECHIKDKEGKAYESVLMISIDELDKMTGTDSNCPNNEPNFFRKHVCDDTKEAAFLNRAARKLKQFLKM |
| | | NISEEFNVHLLTVSQGTQTLVNCTSKEEKNVKEQKKNDACFLKRLLREIKTCWNKILKGSI |
| 41 | Murine IL-21(G₄S)₄-7 | MERTLVCLVVIFLGTVAHKSSPQGPDRLLIRLRHLIDIVEQLKIYENDLDPELLSAPQDVKGHCEHAAFACFQ |
| | | KAKLKPSNPGNNKTFIIDLVAQLRRRLPARRGGKKQKHIAKCPSCDSYEKRTPKEFLERLKWLLQKMIHQHLS |
| | | GPAGGGGSGGGGSGGGGSGGGGSECHIKDKEGKAYESVLMISIDELDKMTGTDSNCPNNEPNFFRKHVCDDTK |
| | | EAAFLNRAARKLKQFLKMNISEEFNVHLLTVSQGTQTLVNCTSKEEKNVKEQKKNDACFLKRLLREIKTCWNK |
| | | ILKGSI |
| 42 | Murine IL-21(G₄S)₄-7 | MERTLVCLVVIFLGTVAHKSSPQGPDRLLIRLRHLIDIVEQLKIYENDLDPELLSAPQDVKGHCEHAAFACFQ |
| | w/o “GPA” | KAKLKPSNPGNNKTFIIDLVAQLRRRLPARRGGKKQKHIAKCPSCDSYEKRTPKEFLERLKWLLQKMIHQHLS |
| | | GGGGSGGGGSGGGGSGGGGSECHIKDKEGKAYESVLMISIDELDKMTGTDSNCPNNEPNFFRKHVCDDTKEAA |
| | | FLNRAARKLKQFLKMNISEEFNVHLLTVSQGTQTLVNCTSKEEKNVKEQKKNDACFLKRLLREIKTCWNKILK |
| | | GSI |
| 43 | Murine IL-21 | MERTLVCLVVIFLGTVAHKSSPQGPDRLLIRLRHLIDIVEQLKIYENDLDPELLSAPQDVKGHCEHAAFACFQ |
| | (EA₃K)₁-7 | KAKLKPSNPGNNKTFIIDLVAQLRRRLPARRGGKKQKHIAKCPSCDSYEKRTPKEFLERLKWLLQKMIHQHLS |
| | | GPAAEAAAKAAAECHIKDKEGKAYESVLMISIDELDKMTGTDSNCPNNEPNFFRKHVCDDTKEAAFLNRAARK |
| | | LKQFLKMNISEEFNVHLLTVSQGTQTLVNCTSKEEKNVKEQKKNDACFLKRLLREIKTCWNKILKGSI |
| 44 | Murine IL-21 | MERTLVCLVVIFLGTVAHKSSPQGPDRLLIRLRHLIDIVEQLKIYENDLDPELLSAPQDVKGHCEHAAFACFQ |
| | (EA₃K)₁-7 | KAKLKPSNPGNNKTFIIDLVAQLRRRLPARRGGKKQKHIAKCPSCDSYEKRTPKEFLERLKWLLQKMIHQHLS |
| | w/o “GPA” | AEAAAKAAAECHIKDKEGKAYESVLMISIDELDKMTGTDSNCPNNEPNFFRKHVCDDTKEAAFLNRAARKLKQ |
| | | FLKMNISEEFNVHLLTVSQGTQTLVNCTSKEEKNVKEQKKNDACFLKRLLREIKTCWNKILKGSI |
| 45 | Murine IL- | MERTLVCLVVIFLGTVAHKSSPQGPDRLLIRLRHLIDIVEQLKIYENDLDPELLSAPQDVKGHCEHAAFACFQ |
| | 21(EA₃K)₅-7 | KAKLKPSNPGNNKTFIIDLVAQLRRRLPARRGGKKQKHIAKCPSCDSYEKRTPKEFLERLKWLLQKMIHQHLS |
| | | GPAAEAAAKEAAAKEAAAKEAAAKEAAAKAAAECHIKDKEGKAYESVLMISIDELDKMTGTDSNCPNNEPNFF |
| | | RKHVCDDTKEAAFLNRAARKLKQFLKMNISEEFNVHLLTVSQGTQTLVNCTSKEEKNVKEQKKNDACFLKRLL |
| | | REIKTCWNKILKGSI |
| 46 | Murine IL- | MERTLVCLVVIFLGTVAHKSSPQGPDRLLIRLRHLIDIVEQLKIYENDLDPELLSAPQDVKGHCEHAAFACFQ |
| | 21(EA₃K)₅-7 | KAKLKPSNPGNNKTFIIDLVAQLRRRLPARRGGKKQKHIAKCPSCDSYEKRTPKEFLERLKWLLQKMIHQHLS |
| | w/o “GPA” | AEAAAKEAAAKEAAAKEAAAKEAAAKAAAECHIKDKEGKAYESVLMISIDELDKMTGTDSNCPNNEPNFFRKH |
| | | VCDDTKEAAFLNRAARKLKQFLKMNISEEFNVHLLTVSQGTQTLVNCTSKEEKNVKEQKKNDACFLKRLLREI |
| | | KTCWNKILKGSI |
| 47 | Murine IL-21R7-FG | MERTLVCLVVIFLGTVAHKSSPQGPDRLLIRLRHLIDIVEQLKIYENDLDPELLSAPQDVKGHCEHAAFACFQ |
| | | KAKLKPSNPGNNKTFIIDLVAQLRRRLPARRGGKKQKHIAKCPSCDSYEKRTPKEFLERLKWLLQKMIHQHLS |
| | | GPAAEAAAKEAAAKEAAAKEAAAKAAAECHIKDKEGKAYESVLMISIDELDKMTGTDSNCPNNEPNFFRKHVC |
| | | DDTKEAAFLNRAARKLKQFLKMNISEEFNVHLLTVSQGTQTLVNCTSKEEKNVKEQKKNDACFLKRLLREIKT |
| | | CWNKILKGSIGPAGGGGSGGGGSGGGGSVSTISSFSPPGYQVSFCLVMVLLFAVDTGLYFSVKTNI |
| 48 | Murine IL-21R7-FG | MERTLVCLVVIFLGTVAHKSSPQGPDRLLIRLRHLIDIVEQLKIYENDLDPELLSAPQDVKGHCEHAAFACFQ |
| | w/o “GPA” | KAKLKPSNPGNNKTFIIDLVAQLRRRLPARRGGKKQKHIAKCPSCDSYEKRTPKEFLERLKWLLQKMIHQHLS |
| | | AEAAAKEAAAKEAAAKEAAAKAAAECHIKDKEGKAYESVLMISIDELDKMTGTDSNCPNNEPNFFRKHVCDDT |
| | | KEAAFLNRAARKLKQFLKMNISEEFNVHLLTVSQGTQTLVNCTSKEEKNVKEQKKNDACFLKRLLREIKTCWN |
| | | KILKGSIGGGGSGGGGSGGGGSVSTISSFSPPGYQVSFCLVMVLLFAVDIGLYFSVKTNI |
| 49 | Murine IL-21R7-RG | MERTLVCLVVIFLGTVAHKSSPQGPDRLLIRLRHLIDIVEQLKIYENDLDPELLSAPQDVKGHCEHAAFACFQ |
| | | KAKLKPSNPGNNKTFIIDLVAQLRRRLPARRGGKKQKHIAKCPSCDSYEKRTPKEFLERLKWLLQKMIHQHLS |
| | | GPAAEAAAKEAAAKEAAAKEAAAKAAAECHIKDKEGKAYESVLMISIDELDKMTGTDSNCPNNEPNFFRKHVC |
| | | DDTKEAAFLNRAARKLKQFLKMNISEEFNVHLLTVSQGTQTLVNCTSKEEKNVKEQKKNDACFLKRLLREIKT |
| | | CWNKILKGSIGPAAEAAAKEAAAKEAAAKEAAAKAAAVSTISSFSPPGYQVSFCLVMVLLFAVDTGLYFSVKT |
| | | NI |
| 50 | Murine IL-21R7-RG | MERTLVCLVVIFLGTVAHKSSPQGPDRLLIRLRHLIDIVEQLKIYENDLDPELLSAPQDVKGHCEHAAFACFQ |
| | w/o “GPA” | KAKLKPSNPGNNKTFIIDLVAQLRRRLPARRGGKKQKHIAKCPSCDSYEKRTPKEFLERLKWLLQKMIHQHLS |
| | | AEAAAKEAAAKEAAAKEAAAKAAAECHIKDKEGKAYESVLMISIDELDKMTGTDSNCPNNEPNFFRKHVCDDT |
| | | KEAAFLNRAARKLKQFLKMNISEEFNVHLLTVSQGTQTLVNCTSKEEKNVKEQKKNDACFLKRLLREIKTCWN |
| | | KILKGSIAEAAAKEAAAKEAAAKEAAAKAAAVSTISSFSPPGYQVSFCLVMVLLFAVDTGLYFSVKTNI |
| 51 | Glycosylphospha- | VSTISSFSPPGYQVSFCLVMVLLFAVDTGLYFSVKTNI |
| | tidylinositol (GPI) | |
| | anchor from human | |
| | CD16b Val196-Ile233 | |
| 52 | Human IL-21(G₄S)₁-7 | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAGGGGSDCDIEGKDGKQYESVLMVSIDQLLDSMKEIGSNCLNNEFNFFKRHICDA |
| | | NKEGMFLFRAARKLRQFLKMNSTGDFDLHLLKVSEGTTILLNCTGQVKGRKPAALGEAQPTKSLEENKSLKEQ |
| | | KKLNDLCFLKRLLQEIKTCWNKILMGTKEH |
| 53 | Human IL-21(G₄S)₁-7 | MRSSPGNMERIVICLMVIFLGTLVHKSSSQGQDRHMIRMRQLIDIVDQLKNYVNDLVPEFLPAPEDVETNCEW |
| | w/o “GAP” | SAFSCFQKAQLKSANTGNNERIINVSIKKLKRKPPSTNAGRRQKHRLTCPSCDSYEKKPPKEFLEREKSLLQK |
| | | MIHQHLSSRTHGSEDSGGGGSDCDIEGKDGKQYESVLMVSIDQLLDSMKEIGSNCLNNEFNFFKRHICDANKE |
| | | GMFLFRAARKLRQFLKMNSTGDFDLHLLKVSEGTTILLNCTGQVKGRKPAALGEAQPTKSLEENKSLKEQKKL |
| | | NDLCFLKRLLQEIKTCWNKILMGTKEH |
| 54 | Human IL-21(G₄S)₄-7 | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAGGGGSGGGGSGGGGSGGGGSDCDIEGKDGKQYESVLMVSIDQLLDSMKEIGSNC |
| | | LNNEFNFFKRHICDANKEGMFLFRAARKLRQFLKMNSTGDFDLHLLKVSEGTTILLNCTGQVKGRKPAALGEA |
| | | QPTKSLEENKSLKEQKKLNDLCFLKRLLQEIKTCWNKILMGTKEH |
| 55 | Human IL-21(G₄S)₄-7 | MRSSPGNMERIVICLMVIFLGTLVHKSSSQGQDRHMIRMRQLIDIVDQLKNYVNDLVPEFLPAPEDVETNCEW |
| | w/o “GAP” | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGGGGSGGGGSGGGGSGGGGSDCDIEGKDGKQYESVLMVSIDQLLDSMKEIGSNCLNN |
| | | EFNFFKRHICDANKEGMFLFRAARKLRQFLKMNSTGDFDLHLLKVSEGTTILLNCTGQVKGRKPAALGEAQPT |
| | | KSLEENKSLKEQKKLNDLCFLKRLLQEIKTCWNKILMGTKEH |
| 56 | Human IL-21(EA₃K)₁-7 | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAAEAAAKAAADCDIEGKDGKQYESVLMVSIDQLLDSMKEIGSNCLNNEFNFFKRH |
| | | ICDANKEGMFLFRAARKLRQFLKMNSTGDFDLHLLKVSEGTTILLNCTGQVKGRKPAALGEAQPTKSLEENKS |
| | | LKEQKKLNDLCFLKRLLQEIKTCWNKILMGTKEH |
| 57 | Human IL-21(EA₃K)₁-7 | MRSSPGNMERIVICLMVIFLGTLVHKSSSQGQDRHMIRMRQLIDIVDQLKNYVNDLVPEFLPAPEDVETNCEW |
| | w/o “GAP” | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSAEAAAKAAADCDIEGKDGKQYESVLMVSIDQLLDSMKEIGSNCLNNEFNFFKRHICD |
| | | ANKEGMFLFRAARKLRQFLKMNSTGDFDLHLLKVSEGTTILLNCTGQVKGRKPAALGEAQPTKSLEENKSLKE |
| | | QKKLNDLCFLKRLLQEIKTCWNKILMGTKEH |
| 58 | Human IL-21(EA₃K)₅-7 | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAAEAAAKEAAAKEAAAKEAAAKEAAAKAAADCDIEGKDGKQYESVLMVSIDQLLD |
| | | SMKEIGSNCLNNEFNFFKRHICDANKEGMFLFRAARKLRQFLKMNSTGDFDLHLLKVSEGTTILLNCTGQVKG |
| | | RKPAALGEAQPTKSLEENKSLKEQKKLNDLCFLKRLLQEIKTCWNKILMGTKEH |
| 59 | Human IL-21(EA₃K)₅-7 | MRSSPGNMERIVICLMVIFLGTLVHKSSSQGQDRHMIRMRQLIDIVDQLKNYVNDLVPEFLPAPEDVETNCEW |
| | w/o “GAP” | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSAEAAAKEAAAKEAAAKEAAAKEAAAKAAADCDIEGKDGKQYESVLMVSIDQLLDSMK |
| | | EIGSNCLNNEFNFFKRHICDANKEGMFLFRAARKLRQFLKMNSTGDFDLHLLKVSEGTTILLNCTGQVKGRKP |
| | | AALGEAQPTKSLEENKSLKEQKKLNDLCFLKRLLQEIKTCWNKILMGTKEH |
| 60 | Human IL-21R7-FG | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAAEAAAKEAAAKEAAAKEAAAKAAADCDIEGKDGKQYESVLMVSIDQLLDSMKEI |
| | | GSNCLNNEFNFFKRHICDANKEGMFLFRAARKLRQFLKMNSTGDFDLHLLKVSEGTTILLNCTGQVKGRKPAA |
| | | LGEAQPTKSLEENKSLKEQKKLNDLCFLKRLLQEIKTCWNKILMGTKEHGPAGGGGSGGGGSGGGGSVSTISS |
| | | FSPPGYQVSFCLVMVLLFAVDTGLYFSVKTNI |
| 61 | Human IL-21R7-FG | MRSSPGNMERIVICLMVIFLGTLVHKSSSQGQDRHMIRMRQLIDIVDQLKNYVNDLVPEFLPAPEDVETNCEW |
| | w/o “GAP” | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSAEAAAKEAAAKEAAAKEAAAKAAADCDIEGKDGKQYESVLMVSIDQLLDSMKEIGSN |
| | | CLNNEFNFFKRHICDANKEGMFLFRAARKLRQFLKMNSTGDFDLHLLKVSEGTTILLNCTGQVKGRKPAALGE |
| | | AQPTKSLEENKSLKEQKKLNDLCFLKRLLQEIKTCWNKILMGTKEHGGGGSGGGGSGGGGSVSTISSFSPPGY |
| | | QVSFCLVMVLLFAVDTGLYFSVKTNI |
| 62 | Human IL-21R7-RG | MRSSPGNMERIVICLMVIFLGTLVHKSSSQGQDRHMIRMRQLIDIVDQLKNYVNDLVPEFLPAPEDVETNCEW |
| | | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSGPAAEAAAKEAAAKEAAAKEAAAKAAADCDIEGKDGKQYESVLMVSIDQLLDSMKEI |
| | | GSNCLNNEFNFFKRHICDANKEGMFLFRAARKLRQFLKMNSTGDFDLHLLKVSEGTTILLNCTGQVKGRKPAA |
| | | LGEAQPTKSLEENKSLKEQKKLNDLCFLKRLLQEIKTCWNKILMGTKEHGPAAEAAAKEAAAKEAAAKEAAAK |
| | | AAAVSTISSFSPPGYQVSFCLVMVLLFAVDTGLYFSVKTNI |
| 63 | Human IL-21R7-RG | MRSSPGNMERIVICLMVIFLGTLVHKSSSQGQDRHMIRMRQLIDIVDQLKNYVNDLVPEFLPAPEDVETNCEW |
| | w/o “GAP” | SAFSCFQKAQLKSANTGNNERIINVSIKKLKRKPPSTNAGRRQKHRLTCPSCDSYEKKPPKEFLERFKSLLQK |
| | | MIHQHLSSRTHGSEDSAEAAAKEAAAKEAAAKEAAAKAAADCDIEGKDGKQYESVLMVSIDQLLDSMKEIGSN |
| | | CLNNEFNFFKRHICDANKEGMFLFRAARKLRQFLKMNSTGDFDLHLLKVSEGTTILLNCTGQVKGRKPAALGE |
| | | AQPTKSLEENKSLKEQKKLNDLCFLKRLLQEIKTCWNKILMGTKEHAEAAAKEAAAKEAAAKEAAAKAAAVST |
| | | ISSFSPPGYQVSFCLVMVLLFAVDTGLYFSVKTNI |

Linker sequences in fusion proteins are underlined.
The “GPA” indicated in bold is not necessary for function.

We claim:

1. A fusion protein comprising (i) interleukin 7 (IL-7) or an IL-7 variant and (ii) IL-21 or and IL-21 variant.

2. The fusion protein of claim 1, wherein the IL-7 is human or murine IL-7.

3. The fusion protein of any one of the preceding claims, wherein the IL-7 or IL-7 variant comprises a sequence that is at least 90% identical to SEQ ID NO:14 or SEQ ID NO:22.

4. The fusion protein of any one of the preceding claims, wherein the IL-7 comprises sequence SEQ ID ID NO: 14 or SEQ ID ID NO:22.

5. The fusion protein of claim 4, wherein the IL-7 comprises SEQ ID ID NO:22.

6. The fusion protein of any one of the preceding claims, wherein the IL-21 is human or murine IL-21.

7. The fusion protein of any one of the preceding claims, wherein the IL-21 or IL-21 variant comprises a sequence that is at least 90% identical to SEQ ID ID NO: 12 or SEQ ID ID NO:20.

8. The fusion protein of any one of the preceding claims, wherein the IL-21 comprises SEQ ID ID NO:12 or SEQ ID ID NO:20.

9. The fusion protein of claim 8, wherein the IL-21 comprises SEQ ID ID NO:20.

10. The fusion protein of any one of the preceding claims, wherein the fusion protein comprises a signal sequence.

11. The fusion protein of claim 10, wherein the signal sequence comprises a sequence that is at least 90% identical to a sequence selected from SEQ ID NOs: 32-35.

12. The fusion protein of claim 11, wherein the signal sequence comprises a sequence selected from SEQ ID NOs: 32-35.

13. The fusion protein of any one of the preceding claims, wherein the IL-7 or IL-7 variant is linked to the IL-21 or IL-21 variant via a linker.

14. The fusion protein of claim 13, wherein the linker is a polypeptide linker.

15. The fusion protein of any one of claims 13-14, wherein the polypeptide linker is a flexible linker.

16. The fusion protein of claim 15, wherein the linker predominantly comprises glycines and serines.

17. The fusion protein of claim 16, wherein the linker comprises SEQ ID NO:27 (GGGS) or one or more repeats of SEQ ID NO:27 (GGGGS).

18. The fusion protein of claim 16, wherein the linker comprises a sequence selected from the group consisting of GGS, SEQ ID NO:28 (GGSGGGS), SEQ ID NO:29 (GGGGGSGGGS), SEQ ID NO:30 (GGGGSGGGSGGGGS), or SEQ ID NO:36 (GGGGGSGGGGSGGGGSGGGGS).

19. The fusion protein of any one of claims 13-14, wherein the polypeptide linker is a rigid linker.

20. The fusion protein of claim 19, wherein the linker predominantly comprises alanines.

21. The fusion protein of claim 19, wherein the linker comprises a sequence that is at least 90% identical to any one of SEQ ID NO:31 (A(EA₃K)₄AAA), SEQ ID NO:37 (A(EA₃K)₁AAA), and SEQ ID NO:38 (A(EA₃K)₅AAA).

22. The fusion protein of claim 21, wherein the linker comprises a sequence selected from the group consisting of SEQ ID NO:31 (A(EA₃K)₄AAA), SEQ ID NO:37 (A(EA₃K)₁AAA), and SEQ ID NO:38 (A(EA₃K)₅AAA).

23. The fusion protein of any one of the preceding claims, wherein the IL-7 or IL-7 variant is located N-terminally of the IL-21 or IL-21 variant.

24. The fusion protein of any one of the preceding claims, wherein the IL-7 or IL-7 variant is located C-terminally of the IL-21 or IL-21 variant.

25. The fusion protein of any one of claims 1-14, wherein the fusion protein comprises a sequence that is at least 90% identical to a sequence selected from of SEQ ID NOs:15-18, 23-26, 39-46, or 52-59.

26. The fusion protein of claim 25, the fusion protein comprises a sequence that is at least 90% identical to a sequence selected from SEQ ID NOs: 23-26 or 52-59.

27. The fusion protein of claim 25, wherein the fusion protein comprises a sequence selected from SEQ ID NOs:15-18, 23-26, 39-46, or 52-59.

28. The fusion protein of claim 27, wherein the fusion protein comprises a sequence selected from SEQ ID NOs: 23-26 or 52-59.

29. The fusion protein ofany one of claims 1-14, wherein the fusion protein comprises a sequence that is at least 90% identical to a sequence selected from SEQ ID NOs:47-50 or 60-63.

30. The fusion protein of claim 29, wherein the fusion protein comprises a sequence that is at least 90% identical to a sequence selected from SEQ ID NOs:60-63.

31. The fusion protein of claim 29, wherein the fusion protein comprises a sequence selected from SEQ ID NOs:47-50 or 60-63.

32. The fusion protein of claim 31, wherein the fusion protein comprises a sequence selected from SEQ ID NOs: 60-63.

33. The fusion protein of any one of the preceding claims, wherein the fusion protein is conjugated to one or more of a cytotoxin, a fluorescent label and an imaging agent.

34. A nucleic acid encoding the fusion protein of any one of the preceding claims.

35. A cell comprising the nucleic acid of claim 34.

36. The cell of claim 35, wherein the cell is a bacterial cell, a yeast cell, an insect cell, or a mammalian cell.

37. The cell of claim 36, wherein the cell is an immune cell.

38. The cell of claim 37, wherein the immune cell is a lymphocyte, a dendritic cell, a natural killer cell, or a macrophage.

39. The cell of claim 37, wherein the cell is a chimeric antigen receptor (CAR) T lymphocyte, a CAR macrophage, a CAR NK, a T cell receptor (TCR) T lymphocyte, or a tumor infiltration lymphocyte.

40. A vector comprising the nucleic acid of claim 34.

41. The vector of claim 40, wherein the vector is a viral vector.

42. The vector of claim 41, wherein the vector comprises a nucleic acid encoding a payload.

43. The vector of claim 42, wherein the payload is a cytokine, a chemokine, a tumor antigen, or a checkpoint inhibitor.

44. The vector of any one of claims 40-43, wherein the viral vector is derived from an oncolytic virus.

45. The vector of claim 44, wherein the oncolytic virus is a vaccinia virus.

46. A cell comprising the vector of any one of claims 40-45.

47. A viral particle comprising the nucleic acid of claim 34.

48. The viral particle of claim 47, the viral particle further comprising a nucleic acid encoding a payload.

49. The viral particle of claim 48, wherein the payload is a cytokine, a chemokine, a tumor antigen, or a checkpoint inhibitor.

50. The viral particle of any one of claims 47-49, wherein viral particle derived from is an oncolytic virus.

51. The viral particle of claim 50, wherein the oncolytic virus is a vaccinia virus.

52. The viral vector of claim 45 or the viral particle of 51 comprising a vaccinia virus genome, wherein the vaccinia virus genome has a deletion of one or more of the thymidine kinase (TK) gene, the vaccinia growth factor (VGF gene), and the A56R gene (coding for hemagglutinin).

53. The viral vector of any one of claim 45 or 52 or the viral particle of any one of claims 51 or 52, wherein the vaccinia virus genome has a deletion of one or more of the viral gene for A41L, A44L, A46R, A49, A52R, A53R, B5R, B8R, BI3R (SPI-2), B15R, B18R, C3L (VCP), C6, C7L, C12L, E3L, F1L, KIL, K3L, K7R, M1L, and NIL.

54. A method for treating cancer, the method comprising administering to a subject in need thereof an effective amount of a fusion protein of any one of claims 1-33 or of a viral particle of any one of claims 47-53.

55. The method of claim 54, wherein the cancer is melanoma, pancreatic cancer, thyroid cancer, lung cancer, colorectal cancer, squamous cancer, prostate cancer, breast cancer, bladder cancer, gastric cancer, sarcoma, mesothelioma, ovarian cancer, endometrial cancer, or cervical cancer.

56. A method for reducing tumor growth, the method comprising administering to a subject in need thereof an effective amount of a fusion protein of any one of claims 1-33 or of a viral particle of any one of claims 47-53.

57. A method for reducing tumor metastasis, the method comprising administering to a subject in need thereof an effective amount of a fusion protein of any one of claims 1-33 or of a viral particle of any one of claims 47-53.

58. A method for increasing cytokine production in the tumor microenvironment, the method comprising administering to a subject in need thereof an effective amount of a fusion protein of any one of claims 1-33 or of a viral particle of any one of claims 47-53.

59. A method for increasing anti-tumor immunity, the method comprising administering to a subject in need thereof an effective amount of a fusion protein of any one of claims 1-33 or of a viral particle of any one of claims 47-53.

60. A method for increasing infiltration of a tumor with immune cells, the method comprising administering to a subject in need thereof an effective amount of a fusion protein of any one of claims 1-33 or of a viral particle of any one of claims 47-53.

61. A method for reducing T cell tolerance, the method comprising administering to a subject in need thereof an effective amount of a fusion protein of any one of claims 1-33 or of a viral particle of any one of claims 47-53.

62. A method for enhancing T cell expansion, the method comprising administering to a subject in need thereof an effective amount of a fusion protein of any one of claims 1-33 or of a viral particle of any one of claims 47-53.

63. A method for increasing the number of memory T cells, the method comprising administering to a subject in need thereof an effective amount of a fusion protein of any one of claims 1-33 or of a viral particle of any one of claims 47-53.

64. The method of any one of claims 54-63, the method further comprising administering to the subject an additional antineoplastic agent.

65. The method of claim 64, wherein the antineoplastic agent is a checkpoint inhibitor, a CART lymphocyte, a CAR macrophage, a CAR NK, a TCR T lymphocyte, or a tumor infiltration lymphocyte.

66. A method of making a fusion protein of any one of claims 1-33, the method comprising
(a) providing a cell expressing the fusion protein of any one of claims 1-33; and
(b) expressing the fusion protein in the cell; and
(c) optionally substantially purifying the fusion protein.


SEQUENCE LISTING (70 sequences)

SEQ ID NO: 1 - DNA, 32 bp, synthetic construct
  source 1..32: mol_type = other DNA
ggcggtcgacatggagaggacccttgtctgtc

SEQ ID NO: 2 - DNA, 34 bp, synthetic construct
  source 1..34: mol_type = other DNA
cgcgggcgcgccctaggagagatgctgatgaatc

SEQ ID NO: 3 - DNA, 32 bp, synthetic construct
  source 1..32: mol_type = other DNA
ccgcggccggcccggagagatgctgatgaatc

SEQ ID NO: 4 - DNA, 37 bp, synthetic construct
  source 1..37: mol_type = other DNA
ggcggtcgacatgttccatgtttcttttagatatatc

SEQ ID NO: 5 - DNA, 38 bp, synthetic construct
  source 1..38: mol_type = other DNA
cgcggttaacttatatactgcccttcaaaattttattc

SEQ ID NO: 6 - DNA, 39 bp, synthetic construct
  source 1..39: mol_type = other DNA
ggcgcctgcaggatgttccatgtttcttttagatatatc

SEQ ID NO: 7 - DNA, 57 bp, synthetic construct
  source 1..57: mol_type = other DNA
ggccggccggccggtggcggtgggagcggtggtgggggttccggaggcggagggtcg

SEQ ID NO: 8 - DNA, 43 bp, synthetic construct
  source 1..43: mol_type = other DNA
ggttccggaggcggagggtcggagtgccacattaaagacaaag

SEQ ID NO: 9 - DNA, 60 bp, synthetic construct
  source 1..60: mol_type = other DNA
ggccggccggccgctgaagctgccgcaaaagaggccgctgcgaaggaggccgcggctaag

SEQ ID NO: 10 - DNA, 60 bp, synthetic construct
  source 1..60: mol_type = other DNA
gaggccgcggctaaggaggcggcagctaaagctgcagccgagtgccacattaaagacaaa

SEQ ID NO: 11 - protein, 146 aa, Mus musculus [sequence omitted]

SEQ ID NO: 12 - protein, 124 aa, Mus musculus
QGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 13 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 14 - protein, 129 aa, Mus musculus
ECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 15 - protein, 293 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAGGGGSGGGGSGGGGSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 16 - protein, 289 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGGGSGGGGSGGGGSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 17 - protein, 302 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAAEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 18 - protein, 299 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSAEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 19 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 20 - protein, 133 aa, Homo sapiens [sequence omitted]

SEQ ID NO: 21 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 22 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 23 - protein, 332 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 24 - protein, 329 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 25 - protein, 341 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAAEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 26 - protein, 337 aa, synthetic construct
RSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 27 - protein, 5 aa, synthetic construct
GGGGS

SEQ ID NO: 28 - protein, 7 aa, synthetic construct
GGSGGGS

SEQ ID NO: 29 - protein, 10 aa, synthetic construct
GGGGSGGGGS

SEQ ID NO: 30 - protein, 15 aa, synthetic construct
GGGGSGGGGSGGGGS

SEQ ID NO: 31 - protein, 24 aa, synthetic construct
AEAAAKEAAAKEAAAKEAAAKAAA

SEQ ID NO: 32 - protein, 22 aa, Mus musculus
MERTLVCLVVIFLGTVAHKSSP

SEQ ID NO: 33 - protein, 25 aa, Mus musculus
MFHVSFRYIFGIPPLILVLLPVTSS

SEQ ID NO: 34 - protein, 29 aa, Homo sapiens
MRSSPGNMERIVICLMVIFLGTLVHKSSS

SEQ ID NO: 35 - protein, 25 aa, Homo sapiens
MFHVSFRYIFGLPPLILVLLPVASS

SEQ ID NO: 36 - protein, 20 aa, synthetic construct
GGGGSGGGGSGGGGSGGGGS

SEQ ID NO: 37 - protein, 9 aa, synthetic construct
AEAAAKAAA

SEQ ID NO: 38 - protein, 29 aa, synthetic construct
AEAAAKEAAAKEAAAKEAAAKEAAAKAAA

SEQ ID NO: 39 - protein, 283 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAGGGGSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 40 - protein, 280 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGGGGSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 41 - protein, 298 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAGGGGSGGGGSGGGGSGGGGSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 42 - protein, 295 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGGGGSGGGGSGGGGSGGGGSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 43 - protein, 287 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAAEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 44 - protein, 284 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSAEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 45 - protein, 307 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAAEAAAKEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 46 - protein, 304 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSAEAAAKEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 47 - protein, 358 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAAEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGPAGGGGSGGGGSGGGGSVSTISSFSPPGYQVSFCLVMVLLFAVDTGLYFSVKTNI

SEQ ID NO: 48 - protein, 352 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSAEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGGGGSGGGGSGGGGSVSTISSFSPPGYQVSFCLVMVLLFAVDTGLYFSVKTNI

SEQ ID NO: 49 - protein, 367 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGPAAEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGPAAEAAAKEAAAKEAAAKEAAAKAAAVSTISSFSPPGYQVSFCLVMVLLFAVDTGLYFSVKTNI

SEQ ID NO: 50 - protein, 361 aa, synthetic construct
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSAEAAAKEAAAKEAAAKEAAAKAAAECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIAEAAAKEAAAKEAAAKEAAAKAAAVSTISSFSPPGYQVSFCLVMVLLFAVDTGLYFSVKTNI

SEQ ID NO: 51 - protein, 38 aa, Homo sapiens
VSTISSFSPPGYQVSFCLVMVLLFAVDTGLYFSVKTNI

SEQ ID NO: 52 - protein, 322 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 53 - protein, 319 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 54 - protein, 337 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 55 - protein, 334 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 56 - protein, 326 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAAEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 57 - protein, 323 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 58 - protein, 346 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAAEAAAKEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 59 - protein, 343 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAEAAAKEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 60 - protein, 397 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAAEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGPAGGGGSGGGGSGGGGSVSTISSFSPPGYQVSFCLVMVLLFAVDTGLYFSVKTNI

SEQ ID NO: 61 - protein, 391 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSVSTISSFSPPGYQVSFCLVMVLLFAVDTGLYFSVKTNI

SEQ ID NO: 62 - protein, 406 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGPAAEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGPAAEAAAKEAAAKEAAAKEAAAKAAAVSTISSFSPPGYQVSFCLVMVLLFAVDTGLYFSVKTNI

SEQ ID NO: 63 - protein, 400 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAEAAAKEAAAKEAAAKEAAAKAAADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHAEAAAKEAAAKEAAAKEAAAKAAAVSTISSFSPPGYQVSFCLVMVLLFAVDTGLYFSVKTNI

SEQ ID NO: 64 - DNA, 53 bp, synthetic construct
  source 1..53: mol_type = other DNA
gatcggccggccggtggcggtgggagcgagtgccacattaaagacaaagaagg

SEQ ID NO: 65 - DNA, 59 bp, synthetic construct
  source 1..59: mol_type = other DNA
gatcggccggccggtggtgggggttcgggtggcggtgggagcggtggtgggggttccgg

SEQ ID NO: 66 - DNA, 57 bp, synthetic construct
  source 1..57: mol_type = other DNA
gatcggccggccgctgaggcggcagctaaagctgcagccgagtgccacattaaagac

SEQ ID NO: 67 - DNA, 60 bp, synthetic construct
  source 1..60: mol_type = other DNA
gatcggccggccgctgaagctgccgctaaggaagctgccgcaaaagaggccgctgcgaag

SEQ ID NO: 68 - DNA, 35 bp, synthetic construct
  source 1..35: mol_type = other DNA
taagctcgaagtcgacatggagaggacccttgtct

SEQ ID NO: 69 - DNA, 46 bp, synthetic construct
  source 1..46: mol_type = other DNA
tcccaccgccaccggccggccctatactgcccttcaaaattttatt

SEQ ID NO: 70 - DNA, 46 bp, synthetic construct
  source 1..46: mol_type = other DNA
cggcagcttcagcggccggccctatactgcccttcaaaattttatt